(19) United States
(12) Patent Application Publication
(10) Pub. No.: US 2004/0057584 A1
(43) Pub. Date: Mar. 25, 2004

(54) Noise control apparatus

(76) Inventors: Isao Kakuhari, Ikoma (JP); Kenichi Terai, Shijonawate (JP); Ko Mizuno, Uji (JP)

Correspondence Address: WENDEROTH, LIND & PONACK, L.L.P., 2033 K STREET N. W., SUITE 800, WASHINGTON, DC, 20006-1021, US

(21) Appl. No.: 10/665,142

(22) Filed: Sep. 22, 2003

(30) Foreign Application Priority Data
Sep. 20, 2002 (JP) 2002-274538
Apr. 2, 2003 (JP) 2003-099066
Jul. 31, 2003 (JP) 2003-283742

(51) Int. Cl. A61F 11/06; G10K 11/16; H03B 29/00

(52) U.S. Cl. 381/71.2; 381/71.7

(57) ABSTRACT

A noise reduction apparatus for reducing noise propagated toward a space 5 on one side of a wall 4 from an external noise source on the other side of the wall 4. The noise reduction apparatus includes a control sound source 1, an error detector 2, and a control section 3. The control sound source 1 is placed on the wall 4 so as to block a noise propagation path. Also, the control sound source radiates a sound into the space 5. The error detector 2 detects the sound propagated from the noise source through the control sound source 1. The control section 3 causes the control sound source 1 to radiate a sound so as to minimize the sound to be detected by the error detector 2 based on the detection results of the error detector 2.

### BACKGROUND OF THE INVENTION

1. Field of the Invention

The present invention relates to a noise reduction apparatus, and more particularly, relates to a noise reduction apparatus performing active noise control.

2. Description of the Background Art

Conventionally, in order to enhance a sound insulation capability of a sound insulation wall, a technique using a heavy material for reducing noise through a wall has been devised. Hereinafter, with reference to FIG. 44, a conventional sound insulation wall will be described.

FIG. 44 is an illustration showing a composite sound insulation material used in the conventional sound insulation wall. In FIG. 44, a composite sound insulation material 81 includes a surface board 82 and a damping material 83. The composite sound insulation material 81 has a structure in which the damping material 83 whose loss coefficient is equal to or greater than 0.2 is laminated on a back side of the surface board 82. Also, the composite sound insulation material 81 is attached to a surface of the sound insulation wall. By the above structured sound insulation wall, vibrations caused by noise are reduced by the damping material 83 having a high loss coefficient, thereby reducing vibrations of the composite sound insulation material 81. As a result, the amount of noise transfer is reduced, whereby a sound insulation capability is enhanced.

Conventionally, a noise reduction apparatus performing active noise control has also been devised. Hereinafter, a conventional noise reduction apparatus will be described with reference to FIGS. 45 to 47.

FIG. 45 is an illustration showing an example of the conventional noise reduction apparatus. In FIG. 45, a sound insulation panel, which is an example of the noise reduction apparatus, includes a sound insulation wall 85, an actuator 86, a vibration sensor 87, a noise detecting sensor 88, a conversion circuit 89, and a control circuit 90. The actuator 86 (represented by a small white circle in FIG. 45) is attached to the sound insulation wall 85 for damping vibrations of the sound insulation wall 85. The vibration sensor 87 (represented by a small black circle in FIG. 45) is also attached to the sound insulation wall 85 for detecting vibrations of the sound insulation wall 85. The conversion circuit 89 calculates a radiation power of sound radiated from the sound insulation wall 85, based on an electrical signal (a signal indicating vibrations of the sound insulation wall 85) output from a plurality of vibration sensors 87. Note that the electrical signals output from all the vibration sensors 87 are input into the conversion circuit 89. However, in FIG. 45, only four vibration sensors 87 on the left side of the insulation wall 85 shown in FIG. 45 are connected to the conversion circuit 89 for the sake of simplicity of the drawing. The noise detecting sensor 88 detects noise transferred through the sound insulation wall 85. The control circuit 90 outputs a control signal for controlling the actuator 86 to the actuator 86, based on outputs of the noise detecting sensor 88 and the conversion circuit 89. Specifically, the control circuit 90 controls the actuator 86 so as to minimize the radiation power of sound, which is calculated by the conversion circuit 89. The above structure allows the sound insulation panel to damp vibrations at a point where the vibration sensor 87 is placed, by the actuator 86. As a result, the amount of noise transfer is reduced, whereby a sound insulation capability is enhanced.

Also, as another example of the noise reduction apparatus performing active noise control, a noise reduction apparatus shown in FIGS. 46 and 47 has been devised. Hereinafter, with reference to FIGS. 46 and 47, the noise reduction apparatus will be described.

FIG. 46 is an illustration showing another example of the conventional noise reduction apparatus. In FIG. 46, a high transmission loss panel 91, which is another example of the noise reduction apparatus, has a structure in which many cells are arranged. Also, FIG. 47 is an illustration showing the detailed structure of a cell 92 shown in FIG. 46. In FIG. 47, the cell 92 includes an actuator 93, a first sensor 94, a second sensor 95, and wall surfaces 97 and 98. Note that, as shown in FIG. 47, the high transmission loss panel 91 includes a control device 96 for each cell. The first sensor 94 is attached to the wall surface 97 of the cell, which faces a noise source (which is placed somewhere in a depth direction of FIG. 47), and detects vibrations of the wall surface 97. The second sensor 95 is attached to a surface of the wall opposite to the first sensor 94, and detects vibrations of the wall surface 98 opposite to the wall surface 97. The actuator 93 is attached to the same side of the second sensor 95.

In the high transmission loss panel 91, the actuator 93 is controlled by the control device 96, based on output signals of the first sensor 94 and the second sensor 95. The control device 96 performs feed forward control based on the output signals of the first sensor 94 and the second sensor 95, thereby controlling the actuator 93. The high transmission loss panel 91 controls vibrations of the wall surface 98 by the above-described method, thereby reducing noise through the cell 92 and enhancing a sound insulation capability.

However, in the above-described conventional sound insulation wall shown in FIG. 44, it is necessary to ensure a high loss coefficient of the damping material 83 in order to achieve sufficient sound insulation for the noise over a wide range of frequencies. That is, as the damping material 83, it is necessary to use a material which is heavy in weight. Thus, in order to support the heavy sound insulation wall, a building in which the insulation wall is installed is required to be solidly constructed.

Also, the actuator generates vibrations in the conventional noise reduction apparatus shown in FIG. 45, whereby an area in the sound insulation wall 85 in which vibrations can be damped is mainly restricted to a portion in which the actuator is placed. Thus, a change in a noise frequency causes a change in a vibration mode of the sound insulation wall 85, thereby causing a change in positions of points, at which vibrations have to be damped on the sound insulation wall 85, and the number thereof. For example, the higher the noise frequency becomes, the more the number of points at which vibrations have to be damped increases. Thus, in order to reduce noise over a wide range of frequencies, a lot of actuators and vibration sensors are required. As a result, there arises a problem of increase in cost and the size of a control circuit for reducing noise over a wide range of frequencies.

Furthermore, in the conventional noise reduction apparatus shown in FIGS. 46 and 47, vibrations of the wall surface are damped on a cell basis. As described above, a change in a noise frequency causes a change in the number of areas on the high transmission loss panel 91 in which vibrations have to be damped. Thus, an adequate size of the cell, and positions of actuators on the cell and the number thereof are changed accordingly with a change in the noise frequency. As a result, it is difficult to control noise over a wide range of frequencies by the noise reduction apparatus shown in FIGS. 46 and 47. Also, in the above-described noise reduction apparatus, there is a possibility that mutual interference between a cell and its adjacent cell produces an undesirable effect. That is, if sound radiated from the actuator of a cell is detected by the sensor of the adjacent cell, a sufficient control effect may not be obtained.

As described above, the conventional technique for active noise reduction has a structure in which the actuator is directly attached to the wall surface whose vibrations have to be damped, whereby it is intrinsically difficult to reduce noise over a wide range of frequencies.

### SUMMARY OF THE INVENTION

Therefore, an object of the present invention is to provide a noise reduction apparatus capable of controlling noise over a wide range of frequencies without increasing the size of the apparatus.

The present invention has the following features to attain the object mentioned above.

A first aspect of the present invention is directed to a noise reduction apparatus for reducing noise propagated toward a predetermined space on one side of a wall from an external noise source on another side of the wall. The noise reduction apparatus comprises a control sound source, a sound detector, and a control section. The control sound source is placed on the wall so as to block a noise propagation path, and radiates a sound into the predetermined space. The sound detector detects a sound propagated from the noise source through the control sound source. The control section causes the control sound source to radiate a sound so as to minimize a sound to be detected by the sound detector, based on the results detected by the sound detector.

Note that the noise reduction apparatus may further comprise a housing, which is attached to the surface of the wall so as to face the noise source, for generating space for noise reduction between the housing and the wall. The control sound source is placed on the housing attached to the surface of the wall. The sound detector is placed in the space for noise reduction. The control sound source radiates a sound into the space for noise reduction.

Also, a plurality of housings may be attached to the surface of the wall adjacently to each other. The noise reduction apparatus further comprises a vibration damping section for damping a vibration in a position of a barycenter of each portion of the surface of the wall, which is divided by the plurality of housings having space for noise reduction.

Note that the vibration damping section may be a pole connecting the housing with the wall. Furthermore, the sound detector may be connected to the pole.

The vibration damping section may be a plummet placed in the position of the barycenter.

Also, the noise reduction apparatus may further comprises a film, which is connected to the housing, for generating a closed space between the film and the control sound source.

Also, the control section may be placed in the space for noise reduction.

Also, the noise reduction apparatus may further comprises a noise detector placed outside the predetermined space for detecting the noise. The control section generates the control signal based on the results detected by the sound detector and the noise detector

Note that the control sound source is typically a piezoelectric loudspeaker.

Also, in a case where the wall has a hole, the control sound source may include a board, a vibrating component, and a driver. The board is connected to the wall so as to block the hole. The vibrating component is placed so as to face the predetermined space for forming an air layer with the board, and being vibrated by a sound radiated into the air layer. The driver radiates the sound into the air layer. The control section causes the driver to radiate the sound by the control signal.

Note that the sound detector is typically placed in the predetermined space, and detects the sound by detecting a sound pressure and a phase of the sound propagated toward the predetermined space.

Note that the sound detector may detect the sound propagated toward the predetermined space by detecting a vibration of the vibrating component.

Also, the board and the vibrating component may be made of a transparent material.

As described above, according to the present invention, it is not necessary to use a heavy material in order to reduce noise over a wide range of frequencies. As a result, a lightweight noise reduction apparatus can be realized. Furthermore, the control sound produced by the control sound source 1 cancels the noise, whereby it is possible to obtain a noise reduction effect over a wide range of frequencies irrespective of frequency of the noise.

Also, in a case where the noise reduction apparatus includes a housing, the present invention can be realized by connecting the housing to the wall. Thus, the noise reduction apparatus can be easily placed.

Also, in a case where the noise reduction apparatus includes a vibration damping section, influences between the adjacent housings can be reduced, thereby designing the control section easily.

Furthermore, in a case where the vibration damping section is a pole, and the sound detector is connected to the pole, it is possible to easily place the sound detector in the space for noise reduction.

Also, in a case where the noise reduction apparatus includes a film, the space for noise reduction can reliably be closed. Thus, it is possible to stabilize a characteristic of the control section, which is set for each space for noise reduction, thereby designing the control section easily.

Also, in a case where the control section is placed in the space for noise reduction, it is possible to enhance weatherability of the control section 3 without the need for a special case. Furthermore, it is possible to place the sound detector and the control section close to each other, thereby reducing an electrical noise interfering with a signal, which is output from the sound detector, while the signal is input into the control section. Thus, it is possible to perform control for the control sound source with further precision, thereby obtaining an excellent noise reduction effect.

Also, in a case where the noise reduction apparatus includes a noise detector, it is possible to perform feed forward control, thereby controlling the control section with further precision.

In a case where the control sound source is a piezoelectric loudspeaker, it is possible to make the control sound source thin and lightweight, thereby realizing a further lightweight noise reduction apparatus.

Also, in a case where the control sound source includes a board, a vibrating component, and a driver, a loudspeaker causing the vibrating component to vibrate by the driver can be applied to the present invention.

Also, in a case where the board and the vibrating component are made of a transparent material, the loudspeaker can be composed by utilizing, for example, a windowpane. As a result, it is possible to place the noise reduction apparatus without causing the user to sense a discomfort at the sight of the loudspeaker on the wall.

These and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is an illustration showing the structure of a noise reduction apparatus according to a first embodiment of the present invention;

FIG. 2 is an illustration showing an apparatus for measuring a capability of a control sound source 1 as a sound insulator;

FIG. 3 is an illustration showing an insertion loss measured by the apparatus shown in FIG. 2;

FIG. 4 is an illustration showing an apparatus for measuring approximation between wavefronts of noise and a control sound;

FIG. 5 is an illustration showing a sound pressure distribution in a range of observation when a noise loudspeaker 7 is activated in the apparatus shown in FIG. 4;

FIG. 6 is an illustration showing a sound pressure distribution in a range of observation when a loudspeaker of the control sound source 1 is activated in the apparatus shown in FIG. 4;

FIG. 7 is an illustration showing a sound pressure distribution in a range of observation when a loudspeaker of a comparison sound source 9 is activated in the apparatus shown in FIG. 4;

FIG. 8 is an illustration showing a phase distribution in a range of observation when the noise loudspeaker 7 is activated in the apparatus shown in FIG. 4;

FIG. 9 is an illustration showing a phase distribution in a range of observation when the loudspeaker of the control sound source 1 is activated in the apparatus shown in FIG. 4;

FIG. 10 is an illustration showing a phase distribution in a range of observation when the loudspeaker of the comparison sound source 9 is activated in the apparatus shown in FIG. 4;

FIG. 11 is an illustration showing a noise reduction characteristic when the comparison sound source 9 is used in the apparatus shown in FIG. 4;

FIG. 12 is an illustration showing a noise reduction characteristic when the control sound source 1 is used in the apparatus shown in FIG. 4;

FIG. 13 is an illustration showing a noise reduction characteristic when the control sound source 1 is used in the apparatus shown in FIG. 4;

FIG. 14 is an illustration showing a variant of the noise reduction apparatus according to the first embodiment;

FIG. 15 is an illustration showing an exemplary detailed structure of a control section 3 shown in FIG. 14;

FIG. 16 is an outline view of a noise reduction apparatus according to a second embodiment;

FIG. 17 is a sectional view in a case where cells shown in FIG. 16 are arranged;

FIG. 18 is an illustration showing a sound insulating effect of having a sound insulating partition between the cells;

FIG. 19 is a sectional view of a case where cells, which are noise reduction apparatuses according to a third embodiment, are arranged;

FIG. 20 is an illustration showing a sound insulating effect by setting a pole;

FIG. 21 is an illustration showing an exemplary variant of the noise reduction apparatus according to the third embodiment;

FIG. 22 is a sectional view of a cell which is a noise reduction apparatus according to a fourth embodiment;

FIG. 23 is an illustration showing transfer functions in a case where cells without a film 27 are attached to a wall;

FIG. 24 is an illustration showing transfer functions in a case where cells with a film 27 are attached to a wall;

FIG. 25 is a sectional view of a cell which is a noise reduction apparatus according to a fifth embodiment;

FIG. 26 is an illustration showing the structure of a noise reduction apparatus according to a sixth embodiment;

FIG. 27 is an illustration showing an effect verification system constructed for verifying a noise reduction characteristic in the sixth embodiment;

FIG. 28 is an illustration showing an effect verification system constructed for verifying a noise reduction effect in the sixth embodiment;

FIG. 29 is an illustration showing a sound pressure distribution of noise over the effect verification system;

FIG. 30 is an illustration showing a phase distribution of the noise over the effect verification system;

FIG. 31 is an illustration showing a sound pressure distribution of a control sound over the effect verification system in a case where a film 36 is formed;

FIG. 32 is an illustration showing a phase distribution of the control sound over the effect verification system in a case where the film 36 is formed;

FIG. 33 is an illustration showing a sound pressure distribution of the control sound over the effect verification system in a case where the film 36 is not formed;

FIG. 34 is an illustration showing a phase distribution of the control sound over the effect verification system in a case where the film 36 is not formed;

FIG. 35 is an illustration showing a distribution of a noise reduction characteristic over the effect verification system in a case where the film 36 is formed;

FIG. 36 is an illustration showing a distribution of the noise reduction characteristic over the effect verification system in a case where the film 36 is formed;

FIG. 37 is an illustration showing a distribution of a noise reduction characteristic over the effect verification system in a case where the film 36 is not formed;

FIG. 38 is an illustration showing a distribution of the noise reduction characteristic over the effect verification system in a case where the film 36 is not formed;

FIG. 39 is an illustration showing the structure of a noise reduction apparatus according to a seventh embodiment;

FIG. 40 is an illustration showing the structure of a noise reduction apparatus according to an eighth embodiment;

FIG. 41 is an illustration showing an exemplary variant of the noise reduction apparatus according to the eighth embodiment;

FIG. 42 is an illustration showing another exemplary variant of the noise reduction apparatus according to the eighth embodiment;

FIG. 43 is an illustration showing the structure of a noise reduction apparatus according to a ninth embodiment;

FIG. 44 is an illustration showing a composite sound insulation material used in a conventional sound insulation wall;

FIG. 45 is an illustration showing an example of a conventional noise reduction apparatus;

FIG. 46 is an illustration showing another example of the conventional noise reduction apparatus; and

FIG. 47 is an illustration showing the detailed structure of a cell 92 shown in FIG. 46.

### DESCRIPTION OF THE PREFERRED EMBODIMENTS

(First Embodiment)

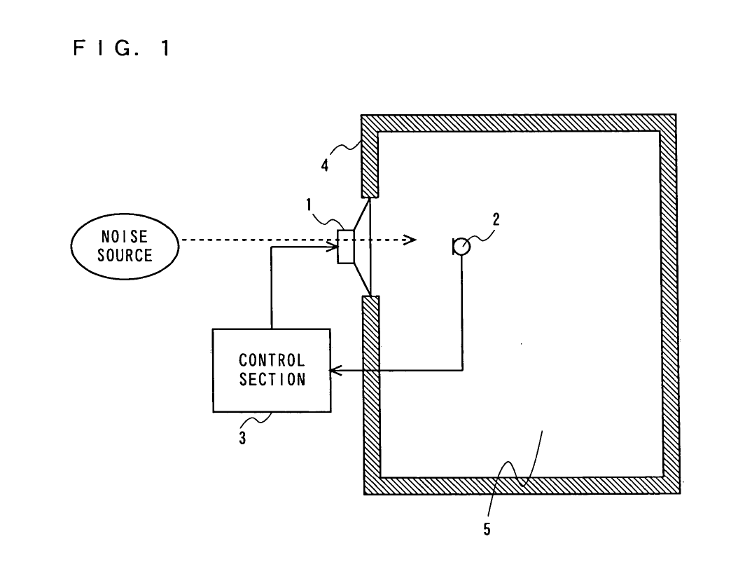

FIG. 1 is an illustration showing the structure of a noise reduction apparatus according to a first embodiment of the present invention. In FIG. 1, the noise reduction apparatus includes a control sound source 1, an error detector 2, and a control section 3. The noise reduction apparatus is placed on a surface of a wall 4 surrounding space 5. The space 5 is space in which noise has to be reduced, and noise enters the space 5 from an external noise source. Here, a path over which an external noise is propagated toward the space 5 is referred to as a noise propagation path. Typically, the noise propagation path passes through a hole of the wall 4 (see a dotted line shown in FIG. 1). However, the noise propagation path is not limited thereto. If there is a portion on the surface of the wall 4 through which noise passes more easily than other portions, a path through the portion may be the noise propagation path. For example, assume that the wall 4 and the space 5 shown in FIG. 1 composes a room in a conventional building and the room has a window, the noise propagation path can be a path through the window.

In FIG. 1, the control sound source 1 is placed so as to block the above-described noise propagation path. Specifically, the wall 4 has a hole, and the control sound source 1 is placed so as to block the hole. In other words, the hole of the wall 4 is used for securing the control sound source 1 thereto. The control sound source 1 is a loudspeaker for canceling the noise in the space 5. The error detector 2 is placed in the space 5. The error detector 2 is a microphone for detecting a sound. The control section 3 is connected to the control sound source 1 and the error detector 2. The control section 3 may be placed in the space 5, or may be placed outside the space 5. Alternatively, the control section 3 may be placed inside of the wall 4.

Next, an operation of the noise reduction apparatus according to the first embodiment will be described. Note that, in following descriptions, it is assumed that noise enters the space 5 surrounded by the wall 4 from the hole in which the control sound source 1 is secured, but not from other portions of the wall 4. Also, it is assumed that a sound in the space 5 is caused only by noise from the outside. In FIG. 1, the error detector 2 detects a sound in the space 5. The detection results are output to the control section 3 as an error signal. Based on the error signal, the control section 3 outputs, to the control sound source 1, a control signal for controlling the control sound source 1. Specifically, the control sound source 1 is controlled so that a sound (noise) in the space 5 becomes zero, that is, the error signal becomes zero. More specifically, the control sound source 1 is controlled so as to produce a sound which is opposite in phase and identical in sound pressure with respect to the noise in a position of the error detector 2. As a result, the control sound source 1 operates so as to cancel the noise propagated toward the space 5 through the noise propagation path.

An operation of the control section 3 will be described in details. In FIG. 1, assume that noise in a position of the error detector 2 is N and a transfer function from the control sound source 1 to the error detector 2 is C, a characteristic of the control section 3 is needed to be set at −1/C. Thus, in the position of the error detector 2, a control sound radiated from the control sound source 1 is calculated as follows:

N·(−1/C)·C=−N

The noise and the control sound from the control sound source 1 interfere with each other, and therefore a sound becomes zero (N+(−N)=0) at the position of the error detector 2. As described above, it is possible to reduce noise in the position of the error detector 2 by causing the noise and the control sound to interfere with each other.

Also, the control sound source 1 is placed on the noise propagation path so as to cancel the noise, and therefore the control sound source 1 itself functions as a sound insulator. FIG. 2 is an illustration showing an apparatus for measuring a capability of the control sound source 1 as a sound insulator. In FIG. 2, the apparatus includes the control sound source 1, a sound tube 6, and a noise loudspeaker 7. In this apparatus, the noise loudspeaker 7 is placed inside the sound tube 6, whose bore is 10 cm, at a closed end thereof, and an electrodynamic loudspeaker, whose bore is 7 cm, is placed at the other end (which is opened) of the sound tube 6 as the control sound source 1. Note that the sound tube 6 is used for preventing the sound produced by the noise loudspeaker 7 from being leaked from an area of the wall other than the area where the control sound source 1 is placed. In the above-described apparatus, the noise loudspeaker 7 is activated (the loudspeaker of the control sound source 1 is not activated), and an insertion loss of a sound is measured using a point 10 cm away from the end of the sound tube 6, at which the control sound source 1 is placed, as an observation point.

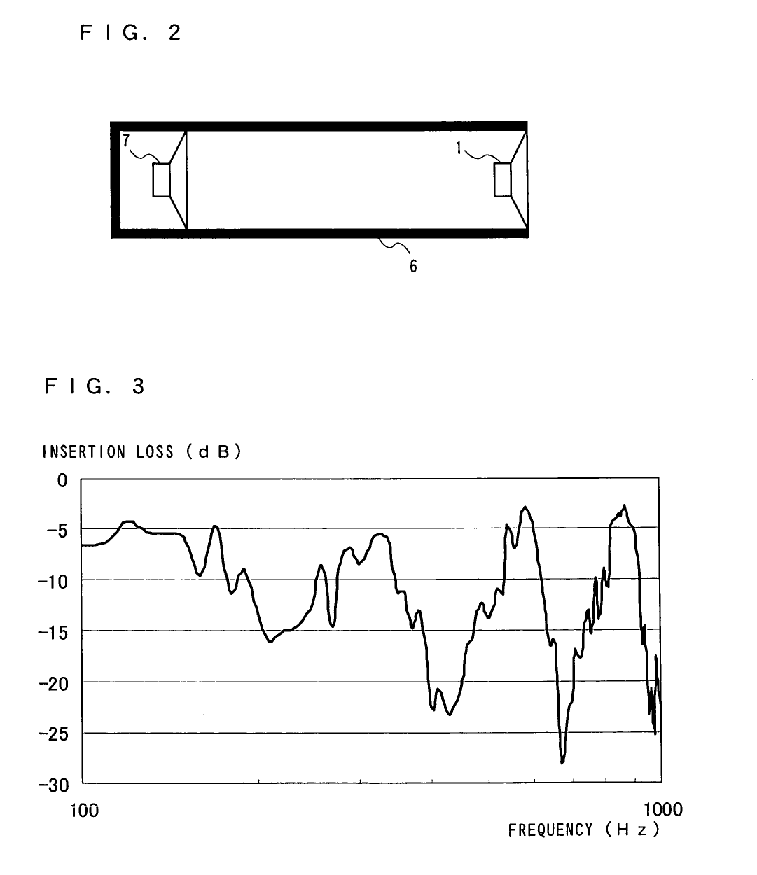

FIG. 3 is an illustration showing an insertion loss measured by the apparatus shown in FIG. 2. FIG. 3 is a graph showing a sound loss in a case where the loudspeaker of the control sound source 1 is inserted, compared to a case where the loudspeaker of the control sound source 1 is not inserted in the sound tube 6, in the apparatus shown in FIG. 2. As shown in FIG. 3, the noise radiated from the sound tube 6 is reduced throughout the observed frequency range by inserting the control sound source 1. Also, −12.1 (dB) is obtained as an average insertion loss in a range from 100 (Hz) to 1 (kHz). Note that the insertion loss varies among frequencies because an acoustic mode occurs in the sound tube 6 due to the control sound source 1 placed at the end of the sound tube 6. As such, the control sound source 1 is placed on the noise propagation path so as to cancel the noise, whereby the control sound source 1 itself cancels the noise. That is, according to the noise reduction apparatus of the first embodiment, active reduction of a sound passing through the wall 4 is realized, and the control sound source 1 itself functions as a sound insulator, whereby it is possible to obtain a further enhanced sound insulation capability.

Furthermore, in the noise reduction apparatus shown in FIG. 1, the noise is propagated toward the space 5 after passing through the control sound source 1. Specifically, the noise is propagated toward the space 5 by vibrations of a diaphragm of the loudspeaker, which is the control sound source 1. On the other hand, as is the case with the noise, the control sound produced by the control sound source 1 for reducing the noise is propagated toward the space 5 by vibrations of the diaphragm of the loudspeaker. Thus, the noise propagated toward the space 5 after passing through the control sound source 1 has a sound wavefront approximated to that of the control sound. Thus, the noise reduction apparatus according to the present invention allows the noise to be reduced over a wide area range in the space 5. Hereinafter, the details will be described.

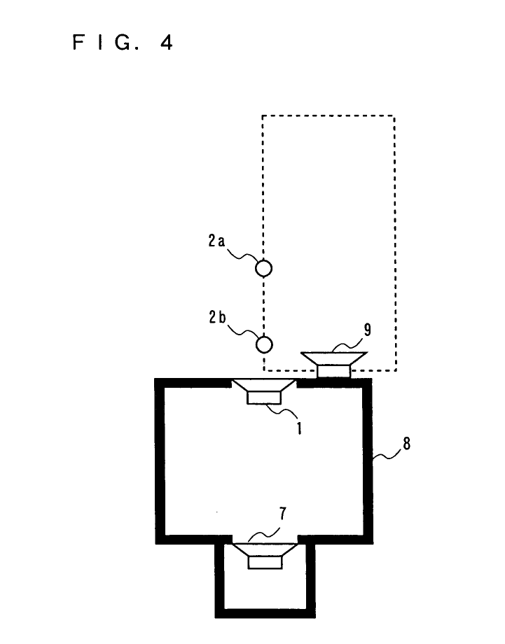

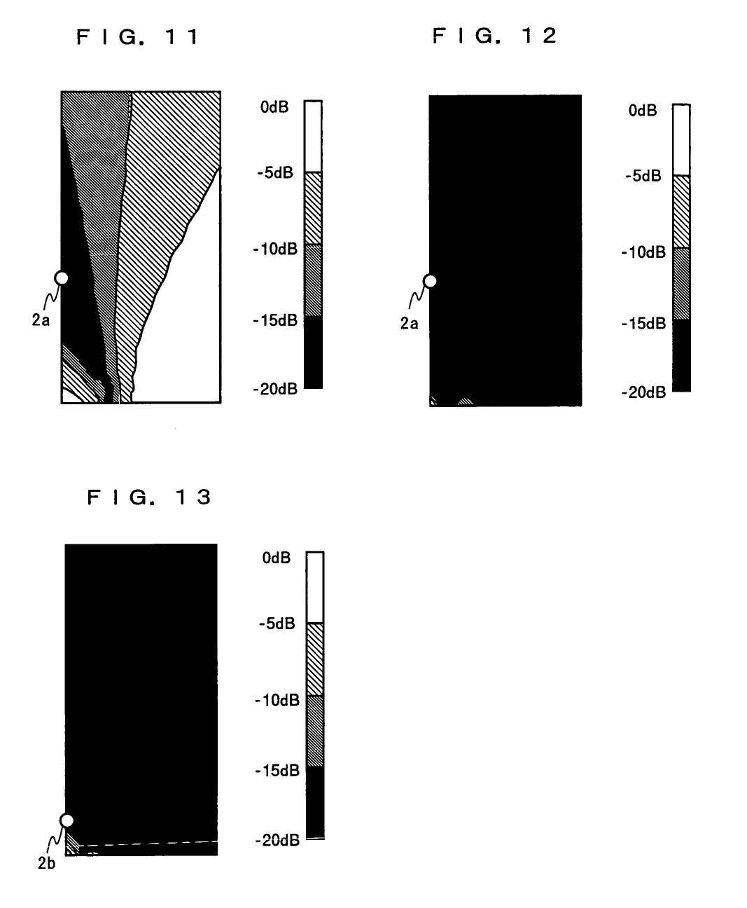

FIG. 4 is an illustration showing an apparatus for measuring approximation between wavefronts of the noise and the control sound. The apparatus shown in FIG. 4 includes the control sound source 1, error detectors 2a and 2b, the noise loudspeaker 7, a soundproof box, and a comparison sound source 9. In this apparatus, the noise loudspeaker 7 is placed on one surface of the soundproof box 8, which is a cube with edges of 30 (cm), and the control sound source 1 is placed on the opposite surface. The control sound source 1 is placed on the noise propagation path, that is, secured in a hole of the soundproof box 8 so as to block the hole. On the other hand, the comparison sound source 9 is placed on a position other than the noise propagation path, that is, on a position other than the hole of the soundproof box 8. The soundproof box 8 is placed so that a sound (noise) produced by the noise loudspeaker 7 is propagated toward the outside of the soundproof box 8 only through the control sound source 1. The error detectors 2a and 2b are used for performing a noise reducing operation. The error detector 2a is placed in a position 20 (cm) away from a center of the soundproof box 8 in a forward (upper portion of FIG. 4)—perpendicular direction. The error detector 2b is placed in a position 5 (cm) away from a center of the soundproof box 8 in a forward-perpendicular direction. The analyzing results in a case where the noise loudspeaker 7, the loudspeaker of the control sound source 1 or a loudspeaker of the comparison sound source 9 are activated in the apparatus shown in FIG. 4 are shown in FIGS. 5 to 10. Also, the analyzing results in a case where the control sound source 1 is activated for canceling the noise and the analyzing results in a case where the comparison sound source 9 is activated for canceling the noise are shown in FIGS. 11 to 13. Note that a dotted line shown in FIG. 4 represents a range of observation, which is shown in FIGS. 5 to 13.

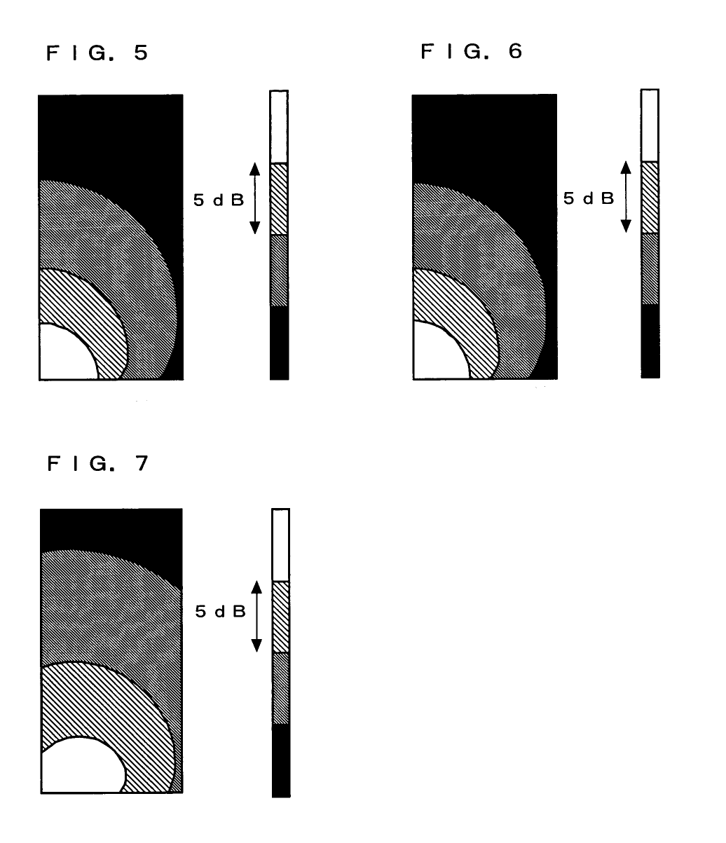

FIG. 5 is an illustration showing a sound pressure distribution in the range of observation when the noise loudspeaker 7 is activated in the apparatus shown in FIG. 4. Also, FIG. 6 is an illustration showing a sound pressure distribution in the range of observation when the loudspeaker of the control sound source 1 is activated in the apparatus shown in FIG. 4, and FIG. 7 is an illustration showing a sound pressure distribution in the range of observation when the loudspeaker of the comparison sound source 9 is activated in the apparatus shown in FIG. 4. As shown in FIGS. 5 and 6, a characteristic of the sound pressure distribution in a case where the noise loudspeaker 7 is activated is extremely similar to the characteristic of the sound pressure distribution in a case where the loudspeaker of the control sound source 1 is activated. On the other hand, FIG. 7 shows that the characteristic of the sound pressure distribution in a case where the loudspeaker of the comparison sound source 9 is activated is different from the characteristic of the sound pressure distribution in a case where the other loudspeaker (i.e., the noise loudspeaker 7 or the loudspeaker of the control sound source 1) is activated.

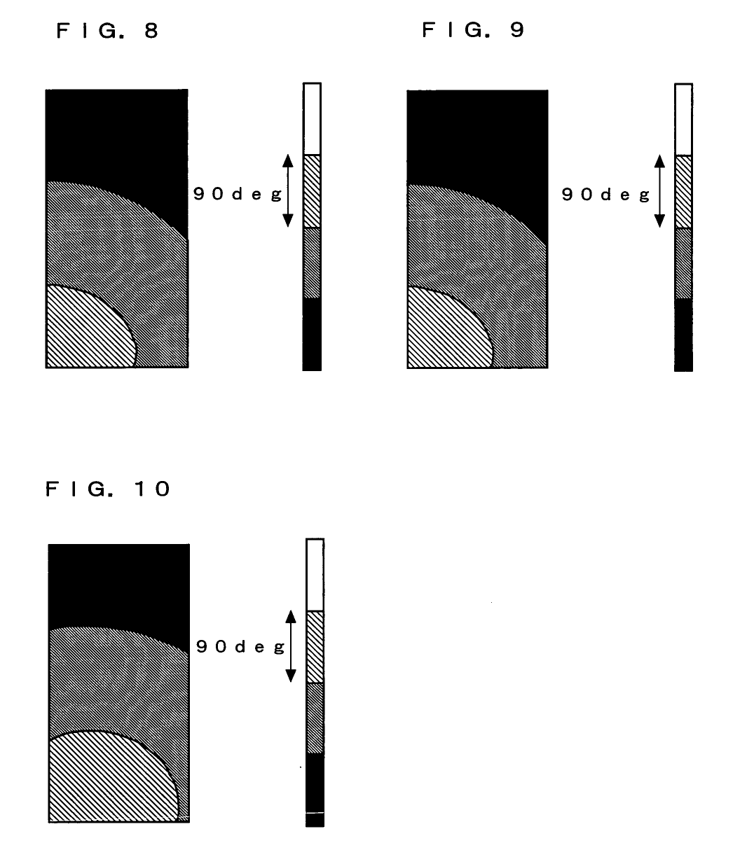

FIG. 8 is an illustration showing a phase distribution in the range of observation when the noise loudspeaker 7 is activated in the apparatus shown in FIG. 4. Also, FIG. 9 is an illustration showing a phase distribution in the range of observation when the loudspeaker of the control sound source 1 is activated in the apparatus shown in FIG. 4, and FIG. 10 is an illustration showing a phase distribution in a range of observation when the loudspeaker of the comparison sound source 9 is activated in the apparatus shown in FIG. 4. As is the case with the sound pressure distribution, FIGS. 8 to 10 show that a characteristic of the phase distribution in a case where the noise loudspeaker 7 is activated is extremely similar to the characteristic of the phase distribution in a case where the loudspeaker of the control sound source 1 is activated, and the characteristic of the phase distribution in a case where the loudspeaker of the comparison sound source 9 is activated is different from the characteristic of the phase distribution in a case where the other loudspeaker (i.e., the noise loudspeaker 7 or the loudspeaker of the control sound source 1) is activated.

According to FIGS. 5 to 10, the control sound and the noise have approximated sound wavefronts. Thus, according to the present invention, it is possible to cause the control sound and the noise to be opposite in phase and identical in sound pressure over a wide area range. As a result, it is possible to obtain a noise reduction effect over a wide area range. Hereinafter, the details will be described using FIGS. 11 to 13.

FIG. 11 is an illustration showing a noise reduction characteristic when the comparison sound source 9 is used in the apparatus shown in FIG. 4. In FIG. 11, among the component elements shown in FIG. 4, the comparison sound source 9 and the error detector 2a are used for reducing the noise produced by the noise loudspeaker 7. Specifically, the comparison sound source 9 is activated so as to minimize the noise in a position of the error detector 2a. On the other hand, FIG. 12 is an illustration showing a noise reduction characteristic when the control sound source 1 is used in the apparatus shown in FIG. 4. In FIG. 12, among the component elements shown in FIG. 4, the control sound source 1 and the error detector 2a are used for reducing the noise produced by the noise loudspeaker 7. Specifically, the control sound source 1 is activated so as to minimize the noise in a position of the error detector 2a.

According to FIG. 11, the use of the comparison sound source 9 allows an enhanced noise reduction effect to be obtained in an area close to the error detector 2a, or in an area extending from the comparison sound source 9 to the position where the error detector 2a is placed, but it is not possible to obtain a noise reduction effect in other areas. The reason is that the sound produced by the comparison sound source 9 and the noise are opposite in phase and identical in sound pressure in the position of the error detector 2a, but not always opposite in phase and identical in sound pressure in other positions, due to different wavefronts of the sound produced by the comparison sound source 9 and the noise. On the other hand, according to FIG. 12, the use of the control sound source 1 allows an enhanced noise reduction effect to be obtained over almost the entire range of observation. The reason is that, if the control sound and the noise are opposite in phase and identical in sound pressure in the position of the error detector 2a, the control sound and the noise are also opposite in phase and identical in sound pressure in other positions due to the approximated wavefronts of the control sound and the noise.

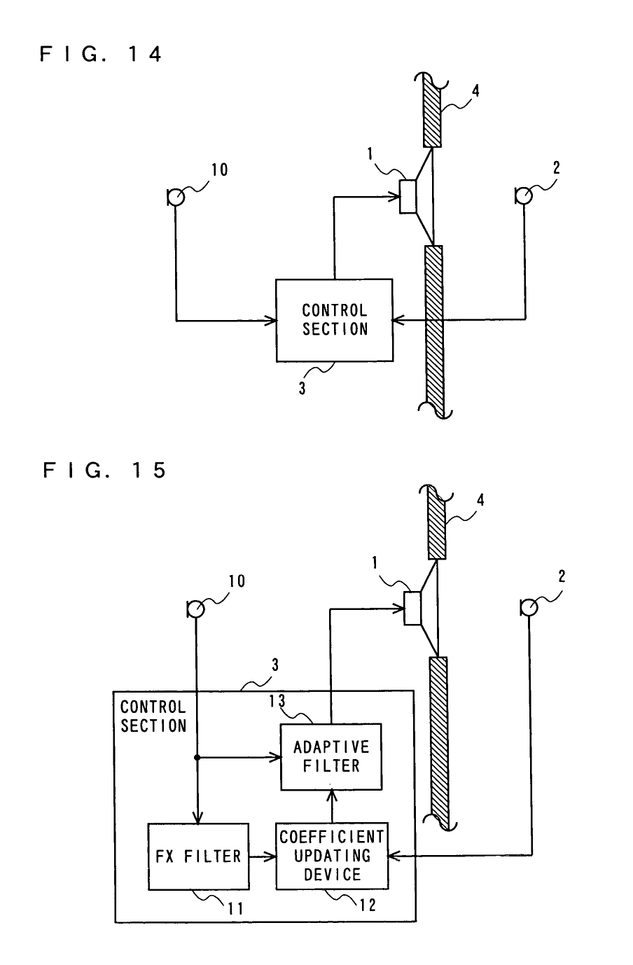

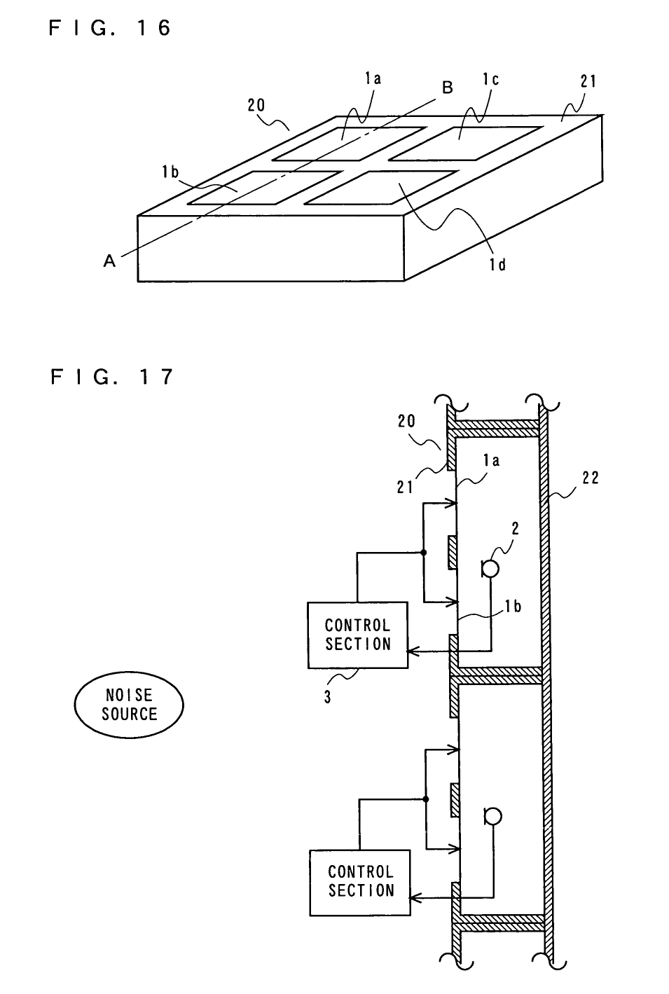

Also, as is the case with FIG. 12, FIG. 13 is an illustration showing a noise reduction characteristic when the control sound source 1 is used in the apparatus shown in FIG. 4. Note that FIG. 13 differs from FIG. 12 in that the error detector 2b is used. That is, FIG. 13 shows the results obtained by activating the control sound source 1 so as to minimize the noise in a position of the error detector 2b. As shown in FIG. 13, in a case where the control sound source 1 is used, it is possible to obtain almost the same noise reduction effect even if a position of the error detector is changed.

As such, according to the present invention, it is not necessary to use a heavy material in order to reduce the noise over a wide range of frequencies, thereby realizing a lightweight noise reduction apparatus. Furthermore, the noise is cancelled by the control sound from the control sound source 1, whereby it is possible to obtain a noise reduction effect over a wide range of frequencies irrespective of frequency of the noise. Also, the control sound source 1 is placed so as to block the noise propagation path, whereby it is possible to cause a wavefront of the control sound to be approximated to a wavefront of the noise. Thus, it is possible to obtain an enhanced noise reduction effect over a wide area range in the space where the noise has to be reduced.

Furthermore, according to the first embodiment, a position of the error detector is not restricted, which is one of the advantages. That is, in a case where the comparison sound source 9 is used for reducing the noise, an enhanced noise reduction effect is obtained only in the vicinity of the error detector, whereby a position of the error detector is restricted to a position where the noise has to be reduced. On the other hand, according to the first embodiment, an enhanced noise reduction effect can be obtained over a wide area range irrespective of a position of the error detector, and therefore a position of the error detector is not restricted. Thus, the noise reduction apparatus according to the first embodiment has more flexibility in design compared to an apparatus using the comparison sound source 9.

Also, according to the first embodiment, it is possible to freely select a position of the error detector, whereby the error detector can be placed in the vicinity of the control sound source. The error detector placed in the vicinity of the control sound source allows the transfer function from the control sound source to the error detector to be minimally affected by a change in an acoustic characteristic (for example, a change in a position of a person or an item, or a change in temperature) of the space where the noise has to be reduced. Thus, according to the first embodiment, if the error detector is placed in the vicinity of the control sound source, an enhanced noise reduction effect can be obtained irrespective of a change in an acoustic characteristic of the space where the noise has to be reduced.

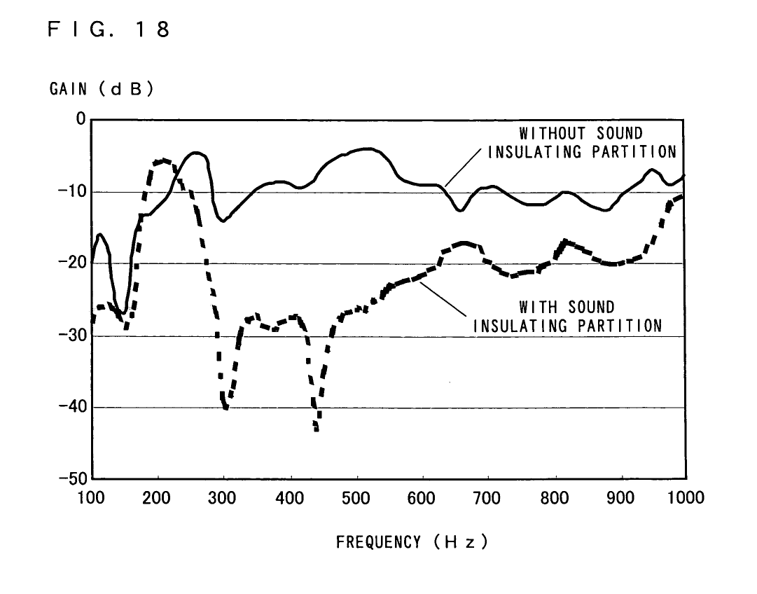

Note that, in the first embodiment, a feedback system for generating a control signal based on an error signal of the error detector 2 is used as the control section 3. In another embodiments, however, a feed forward system may be used as the control section 3 in the noise reduction apparatus. For example, the noise reduction apparatus may have the structure shown in FIG. 14. FIG. 14 is an illustration showing a variant of the noise reduction apparatus according to the first embodiment. The noise reduction apparatus shown in FIG. 14 additionally includes a noise detector 10 along with the component elements shown in FIG. 1. The noise detector 10 is placed outside of the space 5 for detecting noise. In this case, the control section 3 generates a control signal based on the detection results of the error detector 2 and the noise detector 10.

FIG. 15is an illustration showing an exemplary detailed structure of the control section 3 shown in FIG. 14. In FIG. 15, the control section 3 includes an FX filter 11, a coefficient updating device 12, and an adaptive filter 13. The FX filter 11 inputs a signal output from the noise detector 10. A characteristic of the FX filter 11 is set at the same characteristic of the transfer function from the control sound source 1 to the error detector 2. The coefficient updating device 12 inputs the output signal of the error detector 2 as an error input, and inputs a signal output from the FX filter 11 as a reference input. The adaptive filter 13 inputs the signal output from the coefficient updating device 12 and the signal output from the noise detector 10, and outputs a control signal.

In FIG. 15, the coefficient updating device 12 uses Least Mean Square (LMS) algorithm, for example, and performs a calculation for updating a filter coefficient of the adaptive filter 13 so that the error input correlating with the reference input is always minimized. Then, in accordance with the calculation results, the coefficient updating device 12 updates the filter coefficient of the adaptive filter 13. The adaptive filter 13 generates a control signal in accordance with the updated filter coefficient, and outputs the generated control signal to the control sound source 1.

Hereinafter, an operation of the control section 3 of FIG. 15 will be described in further detail. Here, assume that noise in the position of the error detector 2 is N, and a transfer function from the control sound source 1 to the error detector 2 is C. In this case, a characteristic of the FX filter 11 is set at C. The coefficient updating device 12 causes a value of the adaptive filter 13 to converge, thereby bringing a noise component in the output signal of the error detector 2 closer to zero. Then, a value of the adaptive filter 13 is caused to converge to a characteristic −1/C. That is, the output of the adaptive filter 13 becomes N·(−1/C). Thus, the control sound produced by the control sound source 1 becomes N·(−1/C) C in the position of the error detector 2. Then, the noise N, which is to be detected by the error detector 2, is synthesized with the above control sound, and calculated as follows:

N+N·(−1/C)·C=0

The above description shows that the noise is reduced in the noise detector 2.

Note that the control section 3 may have any structure as long as it controls the control sound source 1 so that a sound to be detected by the error detector 2 is minimized. In FIG. 15, the control section 3 performs digital processing using the adaptive filter. However, the control section 3 may be structured using an analog circuit.

Note that, in the first embodiment, the control sound source 1 may be a piezoelectric loudspeaker using a piezoelectric device, or a loudspeaker utilizing another scheme, in place of the above-described electrodynamic loudspeaker. For example, it is possible to obtain the same noise reduction effect also in a case where a loudspeaker radiating a sound by vibrating a board having a vibrator thereon is used as the control sound source.

Note that, in the first embodiment, in a case where there are a plurality of noise propagation paths, a contributing ratio of each noise propagation path (an index showing a ratio of total noise propagated toward the space 5 to the noise propagated over the noise propagation path) may be calculated. In this case, the control sound source is preferably placed so as to block the noise propagation path having the highest contributing ratio.

(second Embodiment)

Next, a noise reduction apparatus according to a second embodiment will be described. Note that, in the noise reduction apparatus according to the first embodiment, the control sound source (loudspeaker) is secured in the hole of the wall. As a result, if the noise reduction apparatus according to the first embodiment is put into practice as it is, the loudspeaker is placed on the wall of a room and fully exposed to view, whereby there is a possibility that a user senses a discomfort at the sight of the loudspeaker on the wall. Thus, in the second embodiment, a noise reduction apparatus having more realistic structure will be offered by applying an operation principle of the present invention.

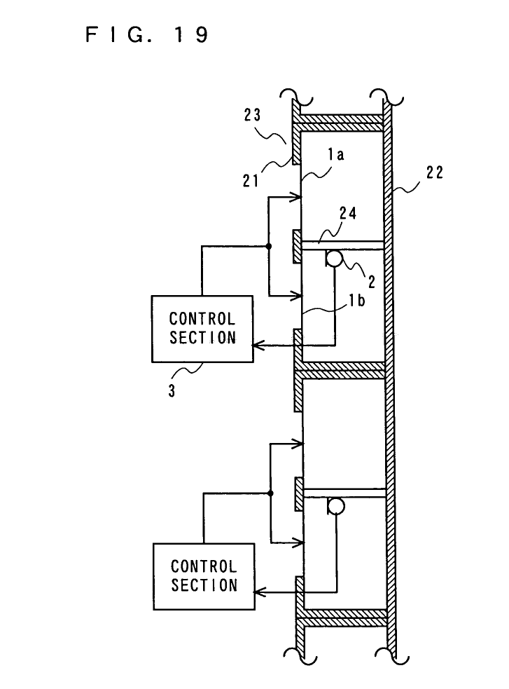

FIG. 16 is an outline view of the noise reduction apparatus according to the second embodiment. The noise reduction apparatus shown in FIG. 16 is structured in units of cells, and a sound insulating panel is structured by arranging a plurality of cells. The sound insulating panel may be structured by bonding individually-made cells to each other, or may be structured by making an integral unit of a plurality of cells. In the second embodiment, the above sound insulating panel is attached to a wall, thereby reducing the noise in the space surrounded by the wall. FIG. 17 is a sectional view in a case where the cells shown in FIG. 16 are arranged. Note that FIG. 17 is a sectional view in a case where the noise reduction apparatus shown in FIG. 16 is sectioned by a line A-B.

In FIGS. 16 and 17, the cell 20 includes four loudspeakers 1a to 1d, the error detector 2, the control section 3, and a housing 21. In the second embodiment, the control sound source is composed of four loudspeakers 1a to 4d. Here, it is assumed that the loudspeakers 1a to 1d are piezoelectric loudspeakers. Note that, in the second and the following embodiments, any component elements that function in similar manners to their counterparts in the first embodiment are denoted by like numerals, with the descriptions thereof omitted.

In FIGS. 16 and 17, the housing 21 is a rectangular parallelepiped whose one surface has holes for securing the loudspeakers 1a to 1d. Note that, in the following descriptions, a surface, which is included in the surfaces of the housing 21, on which the loudspeakers 1a to 1d are secured is referred to as a top surface. The surface opposite to the top surface is opened and attached to a wall 22. Also, the surfaces other than the top surface and the surface opposite thereto are referred to as side surfaces. The loudspeakers 1a to 1d are secured in the holes on the top surface of the housing 21. That is, in the second embodiment, the control sound source is placed on the housing 21 attached to the wall 22. The respective loudspeakers 1a to 1d composing the control sound source can be similar to the loudspeaker of the control sound source 1 in the first embodiment. In FIG. 16, four loudspeakers compose the control sound source, but the number of loudspeakers may be arbitrary. The error detector 2 is placed in the housing 21. The control section 3 is placed in an arbitrary position.

As shown in FIG. 17, the side surfaces of the housing are connected to the side surfaces of other housings, whereby the cells are connected to each other and the sound insulating panel is structured. On each side surface of the housing, a sound insulating partition is set so as to prevent interference of the control sound, which is caused between the adjacent cells. When the housing is attached to the wall, space for noise reduction is formed between the housing and the wall 22. The sound insulating panel is attached to the wall 22 so that the top surface of the housing faces a noise source. That is, in FIG. 17, the space where the noise has to be reduced is on the right side of the wall 22.

Next, an operation of the noise reduction apparatus according to the second embodiment will be described. If it is assumed that the housing 21 is the wall 4 of the first embodiment, and the space surrounded by the cell 20 and the wall 22 is the space 5 of the first embodiment, the noise reduction apparatus according to the second embodiment operates in manners similar to the first embodiment. That is, the control sound produced by the loudspeakers 1a to 1d is applied to the noise propagated toward the inside of the cell through the loudspeakers 1a to 1d and the housing 21. The error detector 2 detects an error sound in the housing 21, and outputs the error sound to the control section 3 as an error signal. The control section 3 generates a control signal based on the error signal, and outputs the control signal to the respective loudspeakers 1a to 1d. More specifically, in FIG. 17, if it is assumed that noise in a position of the error detector 2 is Na, and transfer functions from the loudspeakers 1a to 1d to the error detector 2 are Ca, respectively (transfer functions from the respective loudspeakers 1a to 1d to the error detector 2 are assumed to be the same), a characteristic of the control section 3 is needed to be set at −1/Ca. As a result, in the position of the error detector 2, the control sound radiated from the control sound source is calculated as follows:

Na·(−1/Ca)·Ca=−Na

The noise and the control sound from the control sound source interfere with each other, and therefore a sound becomes zero (Na+(−Na)=0) at the position of the error detector 2. As such, it is possible to reduce the noise in the position of the error detector 2 by causing the noise and the control sound to interfere with each other. Also, as is the case with the first embodiment, it is possible to reduce the noise not only in the position of the error detector 2 but also in almost all the positions in the cell 20.

Next, a case (see FIG. 17) where the sound insulating panel is structured by arranging and connecting a plurality of cells is considered. In this case, there is a possibility that a control sound produced by the control sound source of a cell may affect its adjacent cell. Thus, in the second embodiment, the housing 21 has side surfaces which function as a sound insulating partition, whereby each cell has space where the noise is to be reduced. As a result, the control sound is prevented from being propagated toward the adjacent cell. The above-described structure eliminates the need for considering an undesirable effect of the control sound of the adjacent cell when designing the control section of each cell. Thus, according to the second embodiment, there is an advantage in the control section capable of being structured with a simple circuit. Hereinafter, the above advantage will be described in detail using FIG. 18.

FIG. 18 is an illustration showing a sound insulating effect of having the sound insulating partition between the cells. FIG. 18 shows a difference (gain in FIG. 18) between a level of a sound, produced by the control sound source of a cell and detected by the error detector of the cell, and a level of the sound detected by the error detector of the adjacent cell in the apparatus shown in FIG. 17. Also, a solid line shown in FIG. 18 indicates a case where the sound insulating partition (a side surface of the housing) is used, and a dotted line indicates a case where no sound insulating partition is used. Note that, in FIG. 18, it is assumed that the respective four loudspeakers composing the control sound source are piezoelectric loudspeakers, each measuring 60 mm per side, and the error detector is placed in a position 10 (mm) away from the center of the four loudspeakers in a direction toward the wall 22. Also, the wall 22 is made of an iron plate of 0.5 (mm) in thickness, and the sound insulating partition is made of a resin material of 4 (mm) in thickness, 8 (mm) in height, and 100 (mm) in length.

As shown in FIG. 18, In a case where the sound insulating partition is used, there is a significant difference in sound pressure levels of the two error detectors (the error detector of the cell from which the control sound is produced and the error detector of the adjacent cell) in a wide range of frequencies from 250 (Hz) to 1 (kHz). Note that, in general, in a case where a gain shown in FIG. 18 is smaller than −10 (dB) (that is, in a case where a difference in sound pressure levels is greater than 10 (dB)), there is probably no impact on the adjacent cell. Thus, it is possible to eliminate an undesirable effect on the adjacent cell almost throughout the frequency range by using the sound insulating partition. Note that, in a case where the sound insulating partition is used, there occurs resonance of the wall 22 in a frequency of about 200 (Hz), whereby the difference in sound pressure levels becomes smaller around 200 (Hz). The resonant frequency of the wall 22 varies depending on an area on the surface of the wall divided by the cells, or a material of the wall 22, for example. On the other hand, in a case where no sound insulating partition is used, the difference in sound pressure levels of the two error detectors is smaller compared to a case where the sound insulating partition is used. That is, the control sound produced by the control sound source of a cell enters the error detector of the adjacent cell if there is no sound insulating partition.

Next, a control of the control section in a case where the control sound from the adjacent cell enters the error detector will be considered. The description below examines effects of a control sound from the control sound source of a cell A on a cell B. In the cell B, it is assumed that noise in the position of the error detector is Nb, a transfer function from the control sound source to the error detector is Cb, and a characteristic of the control section is −1/Cb. As aforementioned, the control sound radiated from the control sound source is calculated, in the position of the error detector, as follows:

Nb·(−1/Cb)·Cb=−Nb

Also, the noise and the control sound interfere with each other, and therefore a sound becomes zero (Na+(−Nb)=0) at the position of the error detector. Here, a case where the control sound source of the cell A adjacent to the cell B is activated is considered. The amount of propagation of the control sound of the cell A to the error detector (of the cell B) is assumed to be Da. In this case, the noise, the control sound of the control sound source of the cell B, and the control sound of the control sound source of the cell A interfere with each other in the position of the error detector of the cell B. Thus, a sound in the position of the error detector of the cell B is calculated as follows:

Nb+(−Nb)+Da=Da

That is, the propagation sound Da, which is the control sound from the control sound source of the cell A propagated toward the error detector (of the cell B), becomes a residual noise. Thus, the control sound from the control sound source of the adjacent cell A enters the error detector of the cell B, thereby deteriorating the noise reduction effect. In order to reduce the residual noise Da, it is necessary to set the characteristic of the control section of the cell B at −(Nb+Da)/Cb, which is more complicated compared to a case where the residual noise is zero. Furthermore, considering that the control sound of the control sound source of the cell B is also propagated toward the error detector of the cell A, the characteristic of the control section becomes further complicated in order to obtain a sufficient noise reduction effect in the error detectors of the cell A and the cell B. Also, the above description has shown a case where the two cells are adjacent to each other. However, the more the number of the cells adjacent to each other increases, the more complicated the characteristic of the control section becomes.

As such, if no sound insulating partition is used, the characteristic of the control section becomes very complicated, thereby making it difficult to design the control section. On the other hand, in the second embodiment, the use of the sound insulating partition reduces the control sound propagated from the control sound source of the adjacent cell. As a result, the characteristic of the control section can be set based on the transfer function from the control sound source of a cell to the error detector thereof, thereby simplifying the structure of the control section. Furthermore, the residual noise is reduced, and therefore an excellent noise reduction effect can be obtained.

As described above, in the second embodiment, space for noise reduction is formed between each housing and the surface of the wall 22, and the noise is reduced therein. As a result, the noise is not propagated toward the wall 22, whereby the noise is not propagated toward the space facing an opposite surface of the wall 22 (space on the right side of the wall 22 shown in FIG. 17). Thus, the use of the sound insulating panel shown in FIG. 17 can further reduce the noise.

As described above, according to the second embodiment, it is possible to reduce the noise in the space surrounded by the wall 22 by attaching the noise reduction apparatus on the surface of the wall 22, thereby obtaining the effect similar to the first embodiment. Furthermore, the noise reduction apparatus according to the first embodiment has a restriction that it is required to be secured in the hole of the wall, but the noise reduction apparatus according to the second embodiment does not has such a restriction. Thus, the noise reduction apparatus according to the second embodiment can be easily placed, that is, easily realized, compared to the apparatus according to the first embodiment. For example, it is possible to reduce the noise propagated toward a room by attaching the sound insulating panel on the surface of the wall of the room.

Note that, in the second embodiment, a case where the feedback system in which the control signal is generated based on the error signal output from the error detector is used as a control circuit of the control section has been described. An excellent noise reduction effect can be obtained also in a case where the noise reduction apparatus according to the second embodiment additionally includes the noise detector described in the first embodiment, and the known feed forward system for generating the control signal based on the output signals from the noise detector and the error detector is used as the control circuit. Note that the same can be applied to third to fifth embodiments described below.

(Third Embodiment)

Next, a noise reduction apparatus according to a third embodiment will be described. Note that, in the noise reduction apparatus according to the second embodiment, a sound insulating effect of the sound insulating partition is reduced at a resonant frequency of the wall 22 (see FIG. 18). The noise reduction apparatus according to the third embodiment improves the sound insulating effect of the sound insulating partition at such a frequency.

FIG. 19 is a sectional view of a case where cells, which are noise reduction apparatuses according to the third embodiment, are arranged. A cell 23 shown in FIG. 19 additionally includes a pole 24 along with the component elements included in the cell 20 shown in FIG. 17. Note that the component elements other than the pole 24, which are similar to their counterparts in the cell 20, are denoted by like numerals, with the descriptions thereof omitted. The pole 24 is placed so as to be connected to a center (the vicinity of a barycenter) of each portion of the surface of the wall 22, which is divided by the sound insulating partition.

The noise reduction apparatus according to the third embodiment operates in a manner similar to the noise reduction apparatus according to the second embodiment. Additionally, in the third embodiment, the pole 24 functions as vibration damping means for damping the vibrations of the wall 22. As a result, the vibrations of the wall 22 are damped, whereby it is possible to prevent the control sound from a cell from being propagated toward the error detector of the adjacent cell through the vibrations of the wall 22.

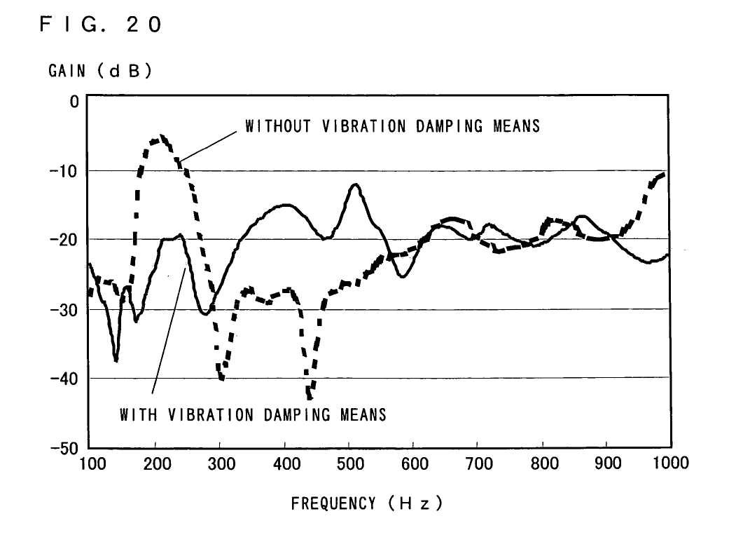

FIG. 20 is an illustration showing a sound insulating effect by setting a pole. FIG. 20 shows a difference (gain in FIG. 20) between a level of a sound produced by the control sound source of a cell and detected by the error detector of the cell, and a level of the sound detected by the error detector of the adjacent cell in the apparatus shown in FIG. 19. Also, a solid line shown in FIG. 20 indicates a case where the pole is used, and a dotted line indicates a case where no pole is used. Note that, in FIG. 20, it is assumed that the pole is a metal pole 5 (mm) in diameter, and it is set so as to connect a center of the top surface of the housing 21 and a center of each portion of the surface of the wall 22 divided by the sound insulating partition. Note that other conditions are similar to those shown in FIG. 18.

FIG. 20 shows that the pole used as the vibration damping means allows a sound pressure level of 20 (dB) to be obtained at a frequency of 200 (Hz) where the sound pressure level is 5 (dB) when no vibration damping means is used. Also, the sound pressure level is reduced at a frequency range from 300 (Hz) to 550 (Hz) compared to a case where no pole is used, but the sound pressure level is at least 10 (dB) throughout the frequency range from 100 (Hz) to 1 (kHz).

As described above, in the noise reduction apparatus according to the second embodiment, a sound insulating effect for an adjacent cell is reduced at a frequency range of 200 (Hz) due to propagation of the control sound produced in a cell to the adjacent cell through the wall 22. More specifically, in the second embodiment, the surface of the wall 22 is divided up into cells (squares measuring 100 (mm) per side), whereby the wall 22 is significantly vibrated at a frequency around 200 (Hz) by the control sound. Then, the vibrations are propagated toward the surrounding adjacent cells, and the error detectors of the adjacent cells detect a radiant sound from the wall 22, which is produced by secondary radiation. Note that, in this case, the strongest vibrations occur in the vicinity of the barycenter of each portion of the surface of the wall 22 divided by the sound insulating partition of each cell.

On the other hand, in the third embodiment, the vibrations of the wall 22 is damped by the vibration damping means, whereby propagation of the vibrations to the surrounding adjacent cells is reduced, and a sound propagated toward the error detectors of the adjacent cells due to the vibrations is also reduced. As a result, a sound insulating effect for the adjacent cell is further enhanced, whereby it is possible to enhance a sound insulation capability of the noise reduction apparatus.

Also, in the third embodiment, the error detector 2 is connected to the pole 24, whereby it is possible to easily place the error detector 2 in the cell 23.

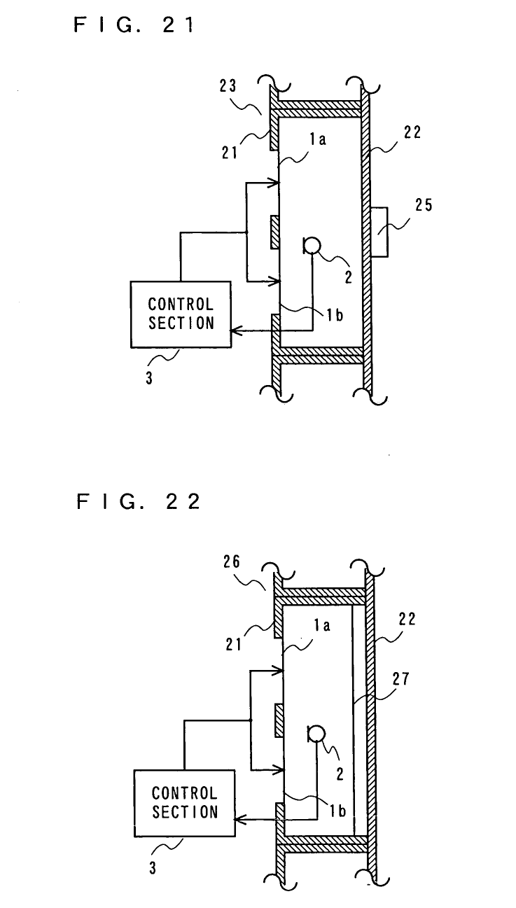

Note that, in the third embodiment, a case where the pole is used as the vibration damping means has been described, but the vibration damping means is not limited thereto. The vibration damping means may be any means as long as an effect of damping the vibrations in the vicinity of the barycenter of each portion of the surface of the wall divided by the sound insulating partition can be obtained. For example, as shown in FIG. 21, a plummet may be used as the vibration damping means. FIG. 21 is an illustration showing an exemplary variant of the noise reduction apparatus according to the third embodiment. Note that FIG. 21 shows only one cell. In FIG. 21, the cell 23 includes a plummet 25 in place of the pole. The plummet 25 is attached to the barycenter of each portion of the surface of the wall 22 divided by the sound insulating partition. As is the case with the pole, the plummet 25 can also damp the vibrations of the wall 22, thereby obtaining the same effect.

(Fourth Embodiment)

Next, a noise reduction apparatus according to a fourth embodiment will be described. Note that, in the above second and third embodiments, there is a possibility that the characteristic of the control section of each cell cannot be stabilized due to irregularities of the surface of the wall (the details will be described below). The fourth embodiment allows the characteristic of the control section of each cell to be stabilized, thereby facilitating a process of designing the control section.

FIG. 22 is a sectional view of a cell which is a noise reduction apparatus according to the fourth embodiment. Note that FIG. 22 shows only a cell 26. In FIG. 22, the cell 26 additionally includes a film 27 along with the component elements included in the cell 20 of FIG. 17. Note that the component elements other than the film 27, which are similar to their counterparts in the cell 20, are denoted by like numerals, with the descriptions thereof omitted. The film 27 is placed so as to block an opening on the side opposite to the top surface of the cell 26. The film 27, the loudspeakers 1a to 1d, and the housing 21 form a closed space.

An operation of the noise reduction apparatus according to the fourth embodiment is similar to the operation of the noise reduction apparatus according to the second embodiment. Thus, if a transfer function from the control sound source to the error detector is assumed to be C, a characteristic of the control section 3 has to be set at −1/C, as mentioned above. That is, in order to perform a precise control, it is preferable to obtain the precise transfer function.

On the other hand, in a case where the noise is reduced using the cell described in the second and the following embodiments, a plurality of cells are required. Thus, it is necessary to determine and set the above-described transfer function with respect to the control section of each cell. Here, the transfer functions may vary among control sections of the cells due to the different attachment status, that is, the irregularities of the surface of the wall 22.

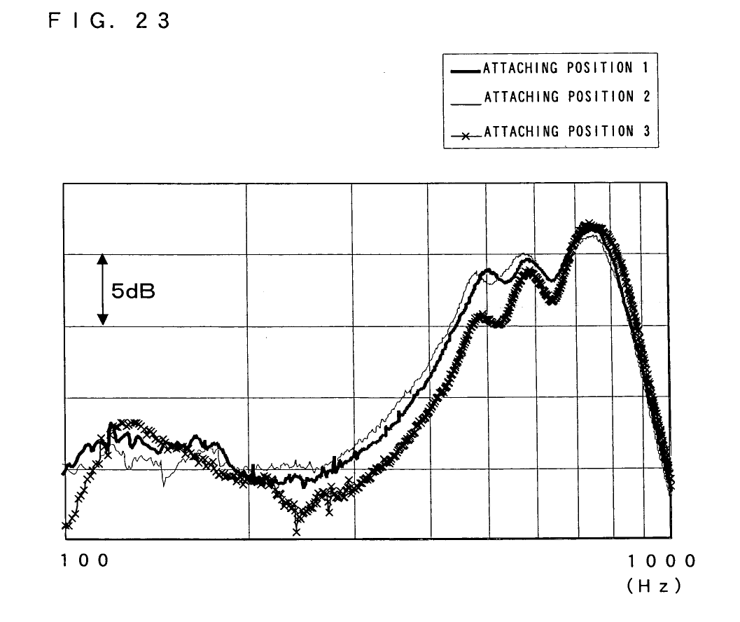

FIG. 23 is an illustration showing transfer functions in a case where cells without the film 27 are attached to the surface of the wall. FIG. 23 shows the results of observing the transfer functions of the identical cells respectively attached to different positions (positions 1 to 3). The transfer functions are assumed to be identical due to the identical cells. However, in the observation results shown in FIG. 23, the characteristics are significantly different especially at a frequency band below 700 (Hz). This is caused by the different attachment status of the cells, that is, the housings 21 of the respective cells are differently attached to the surface of the wall 22 due to the irregularities of the surface of the wall 22. In some attachment positions, the irregularities of the surface of the wall 22 cause a gap to be left between the housing 21 and the surface of the wall 22. The respective cells have different widths of gap. As a result, the respective cells have different degrees of closeness of the space formed by the loudspeakers 1a to 1d, the housing 21, and the surface of the wall 22, which results in a change in impedance of the control sound source composed by the loudspeakers 1a to 1d. For these reasons, the respective cells have different transfer functions.

In a case where each cell has a different transfer function C, it is necessary to adjust the transfer function C of each cell after attaching the cell to the surface of the wall 22, which is a complicated operation. Also, in this case, if a uniform transfer function is set for all the cells, it is impossible to provide a precise transfer function for each cell. As a result, it is impossible to perform a precise control for the control sound source of each cell.

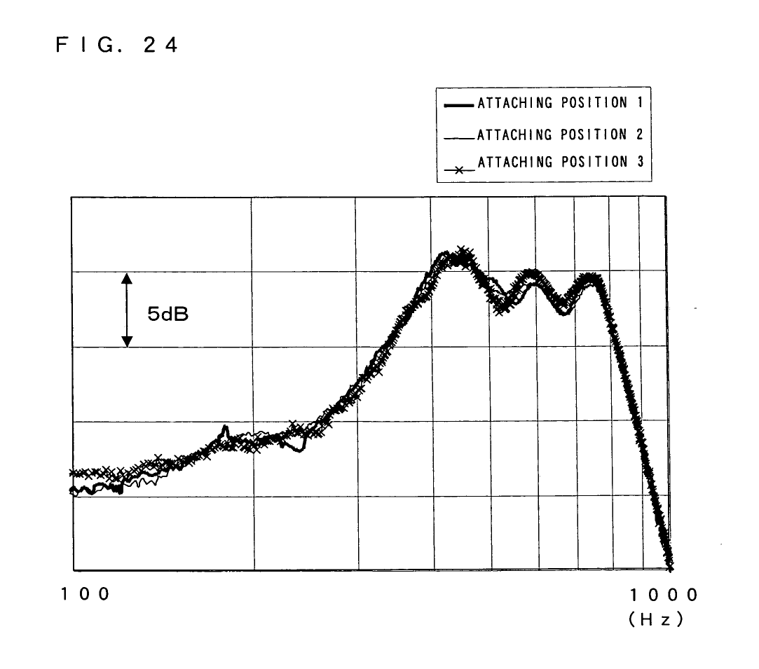

Thus, in the fourth embodiment, the film 27 is formed in the cell 26, thereby forming a closed space in the cell 26. FIG. 24 is an illustration showing transfer functions in a case where cells with the film 27 are attached to the wall. As is the case with FIG. 23, FIG. 24 shows the results of observing the transfer functions of the identical cells respectively attached to different positions (positions 1 to 3). Note that, in this case, it is assumed that the film 27 is a resin film 0.1 (mm) in thickness, and a material of the surface and the positions thereon, to which the cells are attached, are similar to those shown in FIG. 23. In FIG. 23, the three transfer functions are significantly different especially at a frequency band below 700 (Hz). On the other hand, in FIG. 24, the three transfer functions are identical in characteristic throughout the frequency range from 100 (Hz) to 1 (kHz) due to a uniform degree of closeness of the space formed in the cell by the film 27. The three transfer functions are identical in characteristic throughout the above range also because the transfer functions are less affected by the attachment status of the housing 21 to the surface of the wall 22 due to the formation of the above-described space.

As such, according to the fourth embodiment, the transfer function is less affected by the attachment status of the housing 21 to the surface of the wall 22, whereby it is possible to cause the respective cells to have almost uniform transfer functions. Thus, it is possible to set a uniform characteristic in the control section of each cell, thereby facilitating a setting operation of each control section.

Note that, in the fourth embodiment, a case where a film is used has been described, but it is possible to obtain the same effect as the fourth embodiment by stabilizing the degree of closeness of the space in the cell using a plate type component or a component of another shape in place of the film. That is, closed space formation means for forming a closed space in the cell may be a film type component or a plate type component.

Note that the noise reduction apparatus according to the fourth embodiment may additionally include the structure of the third embodiment along with the structure shown in the fourth embodiment. That is, the noise reduction apparatus according to the fourth embodiment may additionally include the pole 24 shown in FIG. 19 or the plummet 25 shown in FIG. 21. As a result, it is possible to obtain the effect described in the third embodiment along with the effect described in the fourth embodiment.

(Fifth Embodiment)

Next, a noise reduction apparatus according to a fifth embodiment will be described. Note that, in the second to fourth embodiments, the control section 3 may be arbitrarily placed. On the other hand, the noise reduction apparatus according to the fifth embodiment specifies a position where the control section has to be placed.

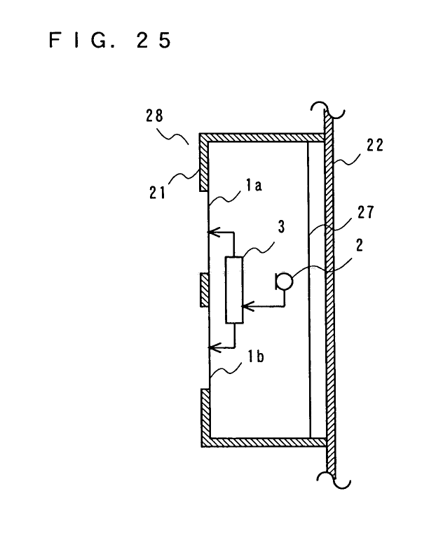

FIG. 25 is a sectional view of a cell which is the noise reduction apparatus according to the fifth embodiment. Note that FIG. 25 shows only a cell 28. The noise reduction apparatus according to the fifth embodiment differs from the noise reduction apparatus according to the fourth embodiment only in that the control section 3 is placed in the cell 28. That is, the control section 3 is placed in a closed space formed by the loudspeakers 1a to 1d, the housing 21, and the film 27. Note that the noise reduction apparatus according to the fifth embodiment operates in similar manners as the noise reduction apparatus according to the fourth embodiment.

The noise reduction apparatus has the following advantage due to the structure shown in FIG. 25. That is, the control section 3 is placed in the closed space, thereby being protected from dust or waterdrops, etc. When the noise reduction apparatus is used, a case is required for protecting the control section 3 from dust or waterdrops, etc. However, according to the fifth embodiment, it is possible to enhance weatherability of the control section 3 without the need for such a case. Furthermore, the error detector 2 and the control section 3 are placed close to each other by placing the control section 3 in the closed space. Thus, it is possible to reduce an electrical noise, which interferes with an error signal output from the error detector 2 while the error signal is input into the control section 3, thereby performing further precise control.

Note that, in FIG. 25, the noise reduction apparatus includes the film 27, but it is possible to obtain the same effect as described above using the structure in which the film 27 is not included. Also, the noise reduction apparatus according to the fifth embodiment may additionally includes the structure of the third embodiment along with the structure shown in the fifth embodiment. As a result, it is possible to obtain the effect described in the third embodiment along with the effect described in the fifth embodiment.

Note that, in the above second to fifth embodiments, the sound in the space formed in the cell is detected using the error detector 2. However, in another embodiment, the sound may be detected by detecting the vibrations of the wall to which the cell is attached. Specifically, vibration detecting means may be placed on the wall to which the cell is attached, thereby performing control by the control section based on the detection results of the vibration detecting means. Also, even in a case where the error detector 2 is used, it is possible to cause the error detector 2 to function as the vibration detecting means by placing the error detector in a position close to the wall.

(Sixth Embodiment)

Next, a noise reduction apparatus according to a sixth embodiment will be described. The noise reduction apparatus according to the sixth embodiment differs from the noise reduction apparatuses described in the second to fifth embodiments, and adopts an operation principle of the first embodiment.

FIG. 26 is an illustration showing the structure of the noise reduction apparatus according to the sixth embodiment. In FIG. 26, the noise reduction apparatus includes the control sound source 1, the error detector 2, and the control section 3. Also, the noise reduction apparatus is placed on a wall 4, which surrounds the space in which noise has to be reduced, so as to block a hole on a noise propagation path of the wall 4, as is the case with the first embodiment.

The noise reduction apparatus according to the sixth embodiment differs from the apparatus according to the first embodiment in the structure of the control sound source 1. In FIG. 26, the control sound source 1 includes a driver 35, a film 36, and a board 37. The board 37 is connected to the wall 4. The board 37 may be a structure separated from the wall 4, or may be a structure united with the wall 4 (that is, a portion of the wall 4 functions as the board 37). The driver 35 is placed in the board 37. The film 36 is formed on one side of the board 37, which is opposite to a noise source. In the sixth embodiment, a loud speaker radiating a sound by vibrating the film 36 by the driver 35 is used as the control sound source 1.

Next, an operation of the noise reduction apparatus according to the sixth embodiment will be described. In the sixth embodiment, noise from the noise source passes through the driver 35 and the board 37 of the control sound source 1, and vibrates the film 36. The vibrations of the film 36 cause the sound to be radiated into the space surrounded by the wall 4, thereby propagating the noise to the error detector 2. On the other hand, the activation of the driver 35 causes air pressure of an air layer 38 to be increased or reduced, whereby the film 36 is vibrated, and a control sound is radiated into the space surrounded by the wall 4.

Operations of the error detector 2 and the control section 3 are the same as the first embodiment. That is, the error detector 2 outputs an error signal to the control section 3. Based on the error signal from the error detector 2, the control section 3 controls the driver 35 so as to minimize the noise to be detected by the error detector 2.

In FIG. 26, the control section 3 includes an FX filter 31, an FB filter 32, a coefficient updating device 33, and an adaptive filter 34. The FX filter 31 inputs the error signal output from the error detector 2. The FX filter 31 has a characteristic equivalent to a transfer function from the driver 35 to the error detector 2. The FB filter 32 inputs a control signal output from the adaptive filter 34. The FB filter 32 has a characteristic similar to that of the FX filter 31, that is, equivalent to the transfer function from the driver 35 to the error detector 2. The coefficient updating device 33 inputs the error signal output from the error detector 2 and a signal output from the FX filter 31. The adaptive filter 34 inputs a signal output from the coefficient updating device 33 and the error signal output from the error detector 2, and outputs the control signal to the driver 35 based on the input signal.

In the control section 3 shown in FIG. 26, a signal output from the FB filter 32 is subtracted from the error signal output from the error detector 2, and the subtraction results are output to the FX filter 31, the coefficient updating device 33, and the adaptive filter 34. The coefficient updating device 33 inputs a signal output from the FX filter 31 as a reference signal. Furthermore, the coefficient updating device 33 performs calculation for updating a filter coefficient of the adaptive filter 34 so that an error input correlating with the reference input is always minimized, in accordance with an LMS algorithm, for example. Then, in accordance with the calculation results, the filter coefficient of the adaptive filter 34 is updated. In accordance with the updated filter coefficient, the adaptive filter 34 generates the control signal, and outputs the generated control signal to the driver 35. Here, if a transfer function from the driver 35 to the error detector 2 is C, the characteristics of the FX filter 31 and the FB filter 32 are set at C, respectively. The FB filter 32 set as described above allows a value of the adaptive filter to converge without producing an oscillation. As a result, the signal corresponding to the noise to be detected by the error detector 2 is brought closer to zero, whereby it is possible to reduce the noise in the vicinity of the error detector 2.

Note that, in FIG. 26, the structure including the FX filter 31, the FB filter 32, the coefficient updating device 33, and the adaptive filter 34 is shown as the detailed structure of the control section 3. However, the control section 3 may be arbitrarily structured as long as the driver 35 is controlled so as to minimize the sound to be detected by the error detector 2.

As shown in the sixth embodiment, the present invention can use a loudspeaker causing the driver 35 to vibrate the film 36 as the control sound source. Also in this case, it is possible to obtain the same effect as the first embodiment. Note that the loudspeaker shown in the sixth embodiment may be composed by utilizing a windowpane, for example (see a ninth embodiment described below). As a result, it is possible to realize the noise reduction apparatus suitable for the use on the wall.

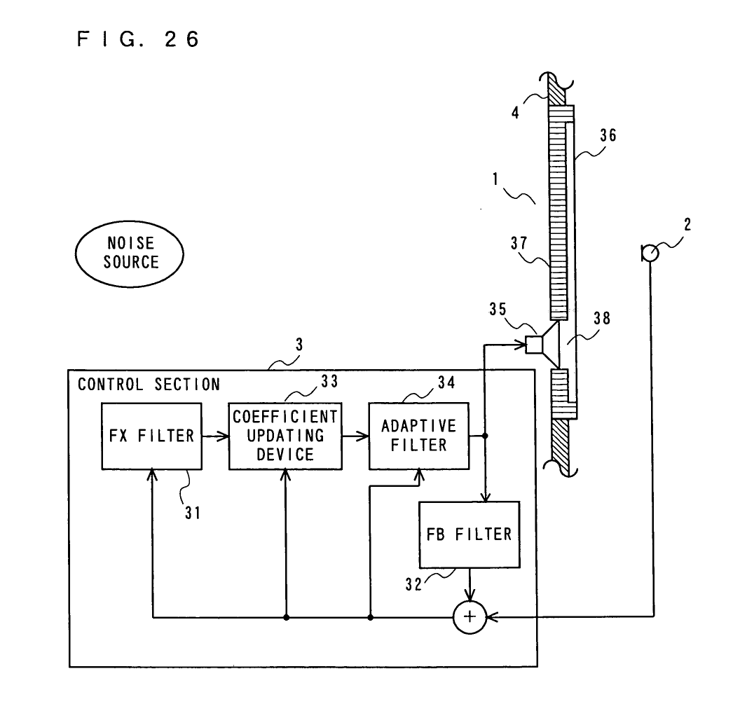

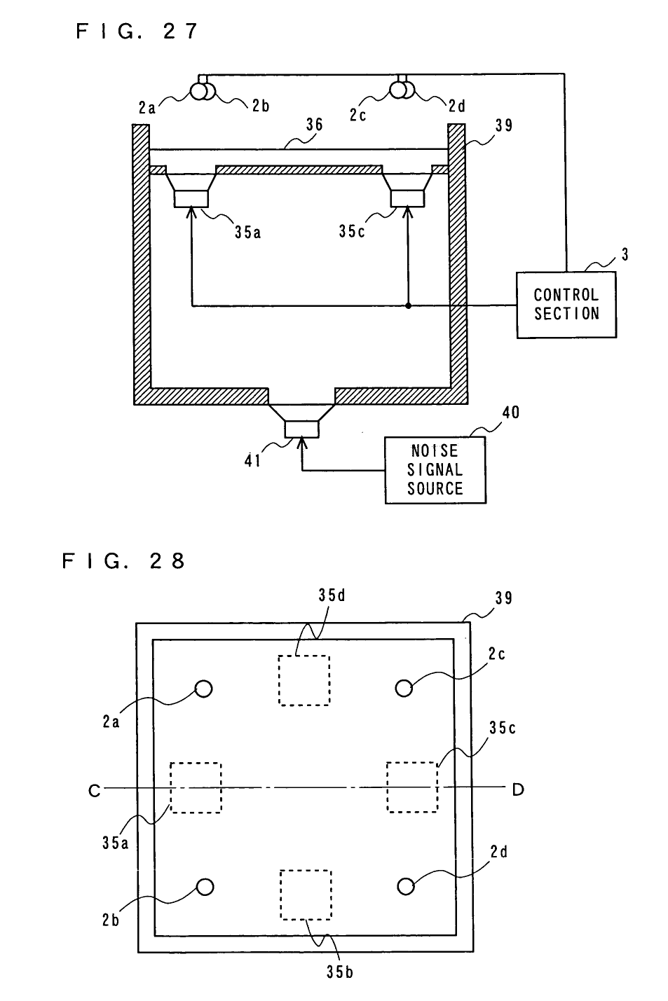

Next, a noise reduction effect by the loudspeaker of the sixth embodiment will be verified. FIGS. 27 and 28 are illustrations showing an effect verification system constructed for verifying a noise reduction effect in the sixth embodiment. FIG. 27 is a vertical sectional view of the effect verification system, and FIG. 28 is a top view of the effect verification system viewed from above (from an upper portion of FIG. 27). Note that FIG. 27 is a sectional view obtained by sectioning the effect verification system shown in FIG. 28 by a line C to D (error detectors 2a to 2d are not on the line C to D, but they are shown in FIG. 27 for facilitating understanding of the invention.

The effect verification system shown in FIGS. 27 and 28 includes four drivers 35a to 35d, four error detectors 2a to 2d, the control section 3, a film 36, a soundproof box 39, a noise source 40, and a noise loudspeaker 41. The soundproof box 39 has sides and a bottom made of a material having a high sound insulation capability. The drivers 35a to 35d are placed on the top surface of the soundproof box 39. Furthermore, the film 36 is formed over the drivers 35a to 35d. The soundproof box 39 has an opening on the top side, and the film 36 is formed so as to block the opening and make a closed space in the soundproof box 39. The noise loudspeaker 41 placed on the bottom of the soundproof box 39 is activated by the noise source 40, thereby radiating noise. The four error detectors 2a to 2d detect the noise passing through the top side of the soundproof box 39 on which the drivers 35a to 35d are placed. Based on the detection results of the four error detectors 2a to 2d, the control section 3 activates the four drivers 35a to 35d, thereby reducing the noise.

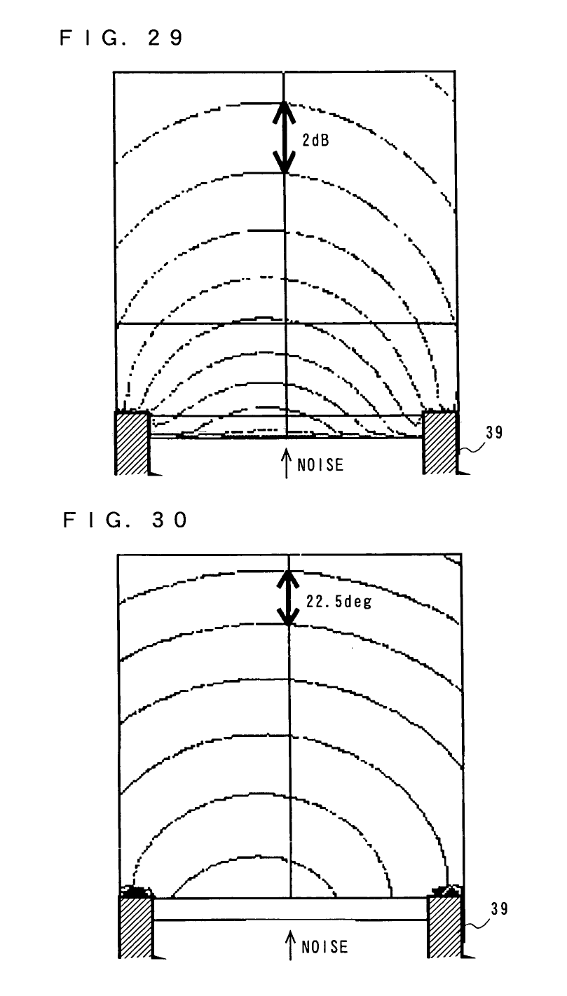

Hereinafter, the observation results obtained by using the above effect verification system are shown in FIGS. 29 to 38. Here, FIGS. 29 to 35, and FIG. 37 show a distribution in a case where the soundproof box is viewed from the side (as shown in FIG. 27). On the other hand, FIGS. 36 and 38 show a distribution in a case where the soundproof box 39 is viewed from above (as shown in FIG. 28). Also, in FIGS. 29 to 35, and FIG. 37, a rectangle in which the distribution is shown is 29 (cm) wide and 32 (cm) long. On the other hand, in FIGS. 36 and 38, a rectangle in which the distribution is shown is 29 (cm) wide and 29 (cm) long.

FIG. 29 is an illustration showing a sound pressure distribution of noise (a sound produced by the noise loudspeaker 41) over the effect verification system. Also, FIG. 30 is an illustration showing a phase distribution of the noise over the effect verification system. In FIGS. 29 and 30, only the noise loudspeaker 41 is activated, and the drivers 35a to 35d, which are the control sound source, are not activated. As shown in FIGS. 29 and 30, the sound pressure and the phase of the noise distribute concentrically around the center of the top side of the effect verification system.

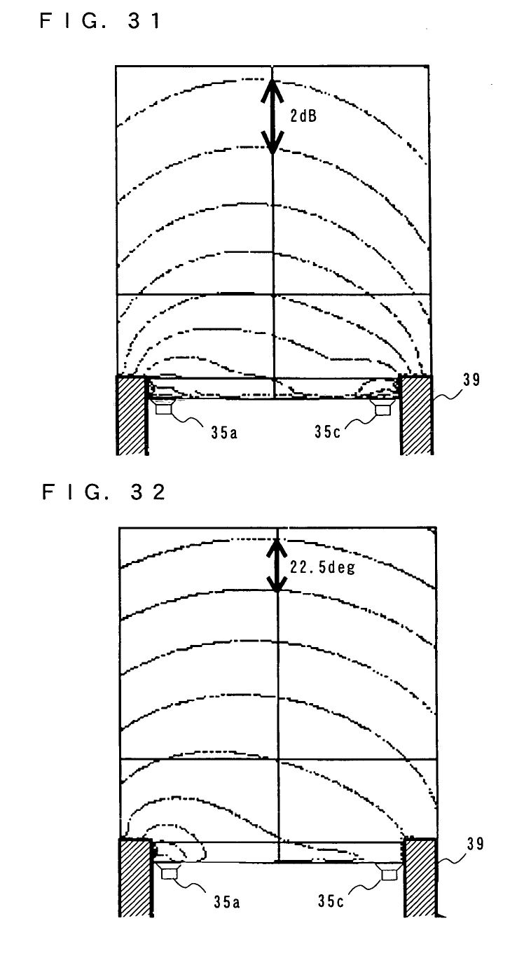

FIG. 31 is an illustration showing a sound pressure distribution of a control sound (a sound produced by the drivers 35a to 35d) over the effect verification system in a case where the film 36 is formed. FIG. 32 is an illustration showing a phase distribution of the control sound over the effect verification system in a case where the film 36 is formed. In FIGS. 31 and 32, only the drivers 35a to 35d, which are the control sound source, are activated, and the noise loudspeaker 41 is not activated. As shown in FIGS. 31 and 32, when the film 36 is formed, the sound pressure and the phase of the control sound distribute in a similar manner as the sound pressure and the phase of the noise.

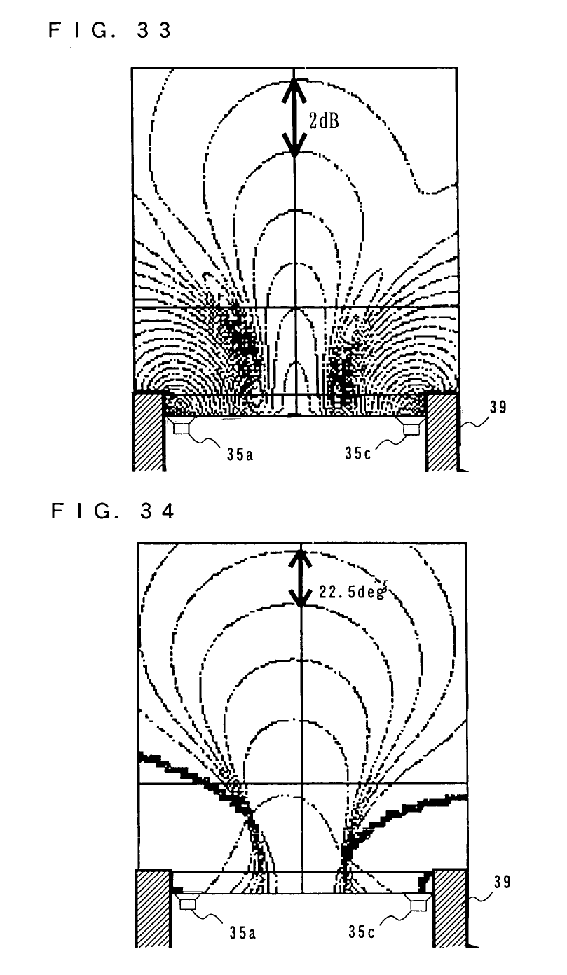

FIG. 33 is an illustration showing a sound pressure distribution of the control sound over the effect verification system in a case where the film 36 is not formed. FIG. 34 is an illustration showing a phase distribution of the control sound over the effect verification system in a case where the film 36 is not formed. In FIGS. 33 and 34, only the drivers 35a to 35d, which are the control sound source, are activated, and the noise loudspeaker 41 is not activated. FIGS. 33 and 34 reveals that the sound pressure and the phase of the control sound distribute in a manner different from the sound pressure and the phase of the noise in a case where the film 36 is not formed.

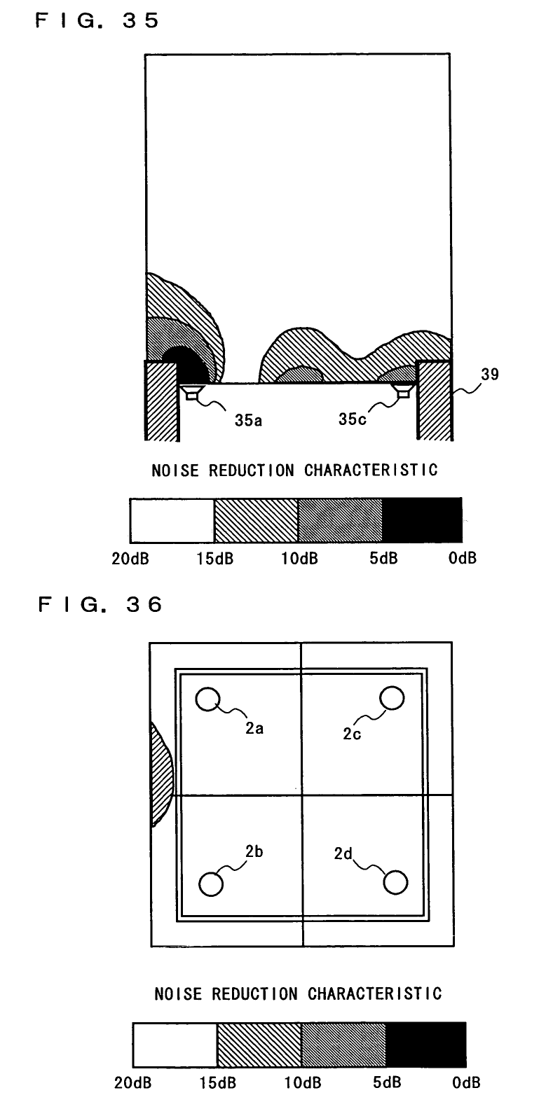

FIGS. 35 and 36 are illustrations showing a distribution of a noise reduction characteristic over the effect verification system in a case where the film 36 is formed. FIGS. 35 and 36 show the noise reduction characteristic in a case where the noise loudspeaker 41 is activated, and the drivers 35a to 35d are also activated so as to minimize a sound to be detected by the error detectors 2a to 2d. FIGS. 35 and 36 reveal that a value of the noise reduction characteristic exceeds 15 (dB) in almost all of the space over the effect verification system in a case where the film 36 is formed.

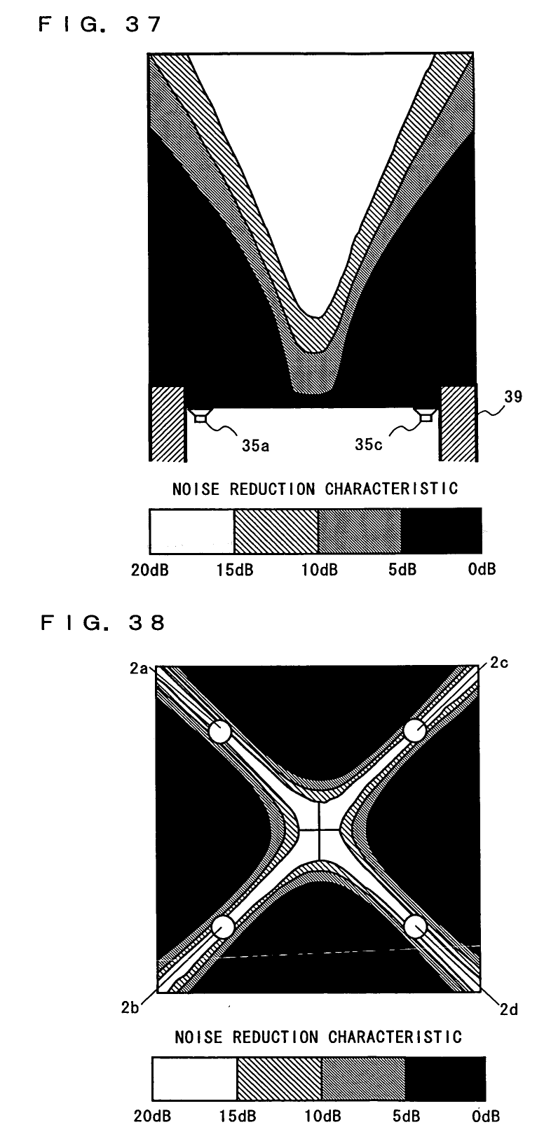

FIGS. 37 and 38 are illustrations showing a distribution of a noise reduction characteristic over the effect verification system in a case where the film 36 is not formed. As is the case with FIGS. 35 and 36, FIGS. 37 and 38 show the noise reduction effect in a case where the drivers 35a to 35d are activated so as to minimize a sound to be detected by the error detectors 2a to 2d. FIGS. 37 and 38 reveal that a sufficient noise reduction effect is obtained only in the vicinity of the error detectors 2a to 2d in a case where the film 36 is not formed.

As described above, the formation of the film 36 allows a sufficient noise reduction effect to be obtained not only in the vicinity of the error detector but also in a further wide area.

Note that, in the sixth embodiment, the structure having the film 36 has been described. However, a vibrating material vibrated by the driver is not limited to a transparent film. Any vibrating component may be used as long as it is placed so that an air layer is formed between the component and the board, and as long as it is placed so as to be capable of being vibrated by the sound radiated by the air layer. For example, a transparent board, which is used in place of the film 36, is connected to the board by a suspension of elastic body. The above structure allows the transparent board to be vibrated by the driver, whereby it is possible to use the transparent board as the vibrating material.

(Seventh Embodiment)

Next, a noise reduction apparatus according to a seventh embodiment will be described. The noise reduction apparatus according to the seventh embodiment causes the control section to perform feed forward control.

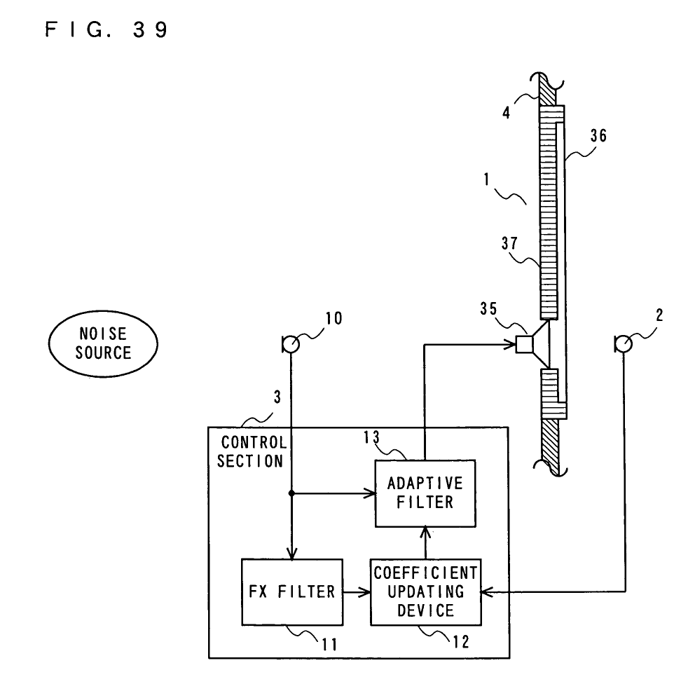

FIG. 39 is an illustration showing the structure of the noise reduction apparatus according to the seventh embodiment. In FIG. 39, the noise reduction apparatus includes the noise detector 10 along with the component elements of the noise reduction apparatus according to the sixth embodiment. Note that the noise detector 10 is the same as that shown in FIG. 16. Also, the control section 3 has the same structure as that shown in FIG. 16. Thus, detailed descriptions of an operation of the seventh embodiment are omitted.

As such, even in a case where the loudspeaker vibrating a film by a driver is used as the control sound source, it is possible to cause the control section 3 to perform feed forward control. As a result, it is possible to control the driver with further precision. Also in the seventh embodiment, it is possible to obtain the same effect as the sixth embodiment.

(Eighth Embodiment)

Next, a noise reduction apparatus according to an eighth embodiment will be described. In the noise reduction apparatus according to the eighth embodiment, a sound propagated toward space is detected by vibrations of a film.

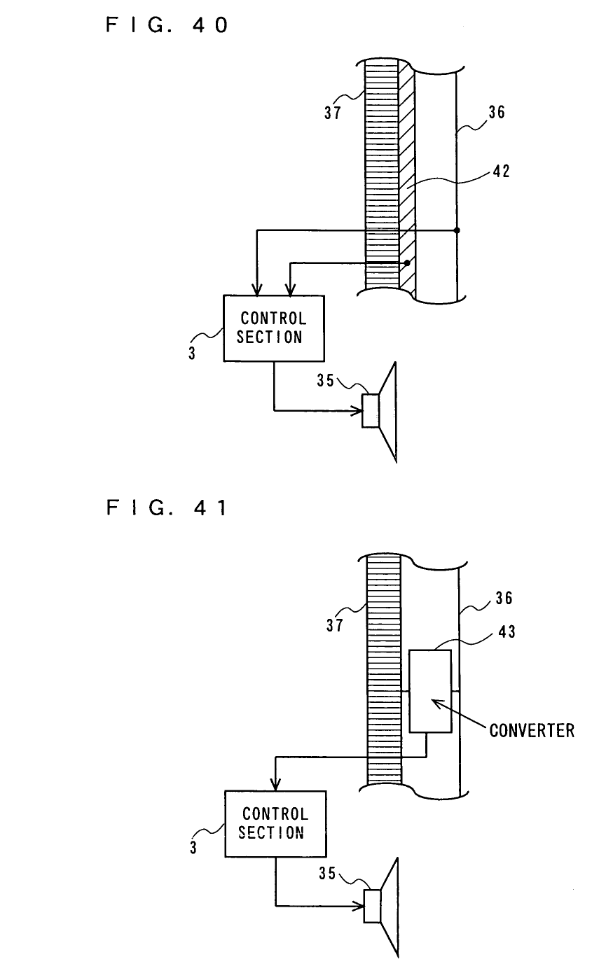

FIG. 40 is an illustration showing the structure of the noise reduction apparatus according to the eighth embodiment. Note that the entire structure of the noise reduction apparatus according to the eighth embodiment is the same as that of the sixth embodiment. Thus, in FIG. 40, a portion different from the sixth embodiment is mainly shown. In FIG. 40, the noise reduction apparatus includes a back plate 42 along with the component elements shown in FIG. 26. Note that the noise reduction apparatus does not include the error detector 2. The back plate 42 is attached to a surface of the board 37, which faces the film 36.

In the eighth embodiment, static electricity is built up between the film 36 and the back plate 42 by charging the film 36, thereby forming a condenser. Note that, in the eighth embodiment, an electret material is preferably used, that is, a high polymer material, such as polypropylene, Teflon (R), or polyethylene, etc., having a permanent polarization or fixed charge, as the film 36. The above structure allows a capacitance of the condenser to be changed with a change in a distance between the film 36 and the back plate 42, which is caused by the vibrations of the film 36, whereby a signal indicating the vibrations of the film 36 is output to the control section 3. This signal corresponds to the above-described error signal. As such, it is possible to detect a sound radiated into the space by detecting the vibrations of the film 36. Note that operations of the control section 3 and the driver 35 are the same as those described in the sixth embodiment.

As such, the eighth embodiment uses the structure by which the sound radiated into the space is detected by detecting the vibrations of the film 36 in place of detecting a sound pressure and a phase by the error detector 2. According to the above structure, it is also possible to obtain the same effect as the sixth embodiment.

Also, as another structure in which the vibrations of the film 36 are detected, the structure shown in FIG. 41 can be possible. FIG. 41 is an illustration showing an exemplary variant of the noise reduction apparatus according to the eighth embodiment. The noise reduction apparatus shown in FIG. 41 includes a converter 43 for outputting a vibration signal by detecting the vibrations of the film 36. The control section 3 uses the vibration signal as an error signal. According to the above structure, it is also possible to obtain the same effect as the sixth embodiment.

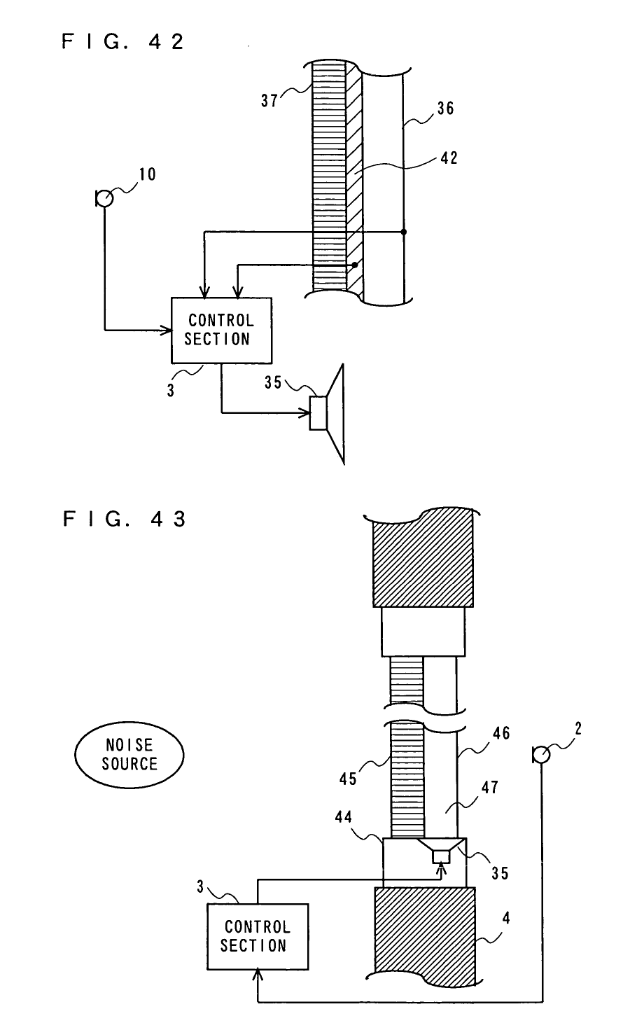

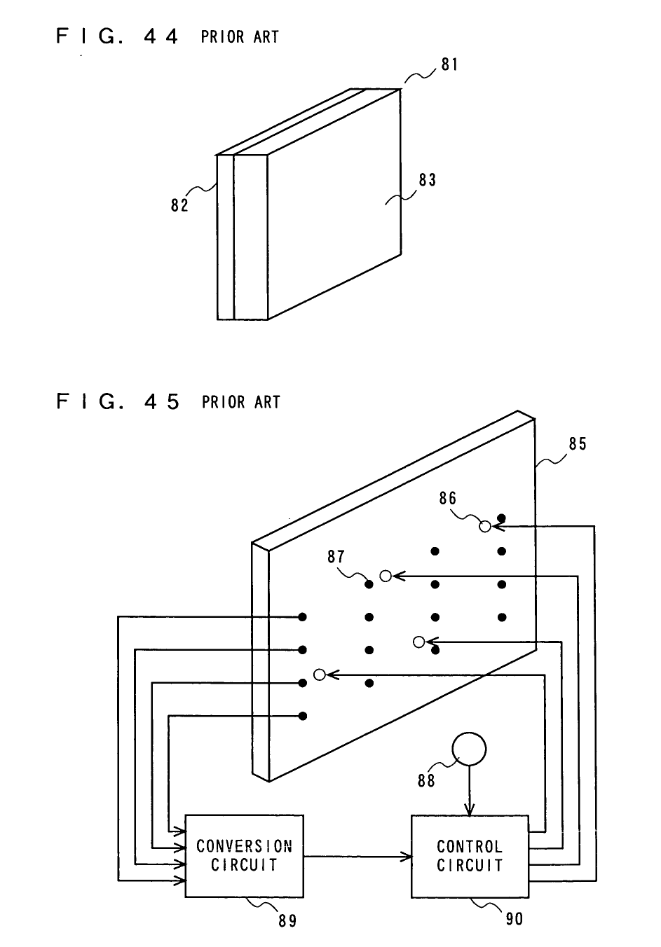

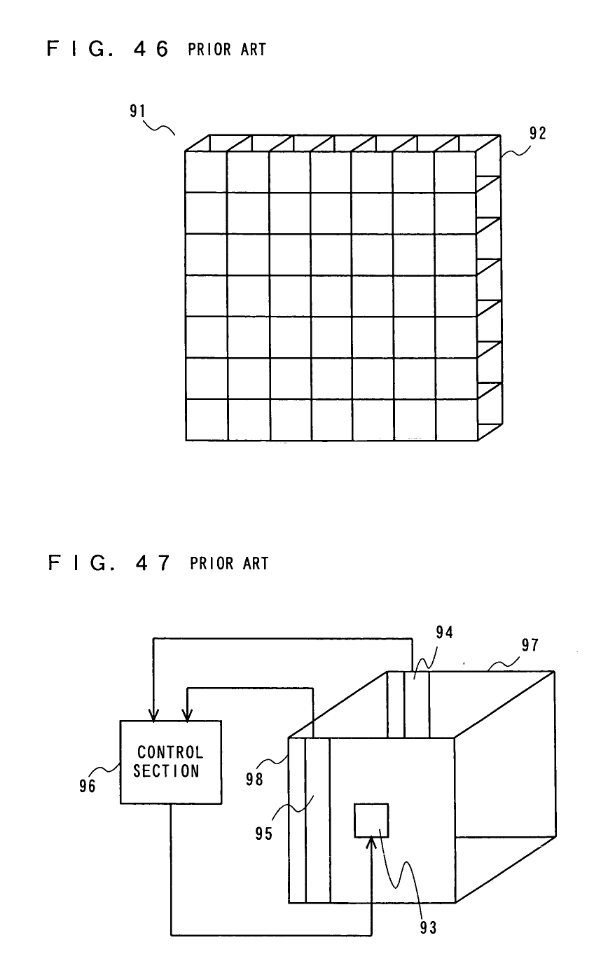

Also, FIG. 42 is an illustration showing another exemplary variant of the noise reduction apparatus according to the eighth embodiment. As shown in FIG. 42, the eighth embodiment may additionally include the noise detector 10, as is the case with the other embodiments.

(Ninth Embodiment)

Next, a noise reduction apparatus according to the ninth embodiment will be described. In the ninth embodiment, a loudspeaker, which is the control sound source, is composed utilizing a windowpane. As a result, it is possible to realize the noise reduction apparatus suitable for use on a wall.

FIG. 43 is an illustration showing the structure of the noise reduction apparatus according to the ninth embodiment. In FIG. 43, the noise reduction apparatus includes the error detector 2, the control section 3, the driver 35, a sash 44, a glass 45, and a transparent film 46. The sash 44 is built into the wall 4, and the glass 45 is installed into the sash 44. The transparent film 46 is formed so as to face the noise source across the glass 45. The transparent film 46 is formed so that an air layer 47 is formed between the transparent film 46 and the glass 45. The driver 35 is built into the sash 44 so as to radiate a sound into the air layer 47.

In FIG. 43, the glass 45 and the sash 44 correspond to the board 37 shown in FIG. 26. Also, the transparent film 46 corresponds to the film 36 shown in FIG. 26. Thus, in the ninth embodiment, the control sound source is composed of the driver 35, the sash 44, the glass 45, and the transparent film 46. That is, the loudspeaker, which is the control sound source, is composed utilizing the windowpane. The loudspeaker of the ninth embodiment can radiate a sound, as is the case with the sixth embodiment, by causing the driver 35 to vibrate the transparent film 46. Also, operations of the error detector 2 and the control section 3 are the same as those in the sixth embodiment. As a result, the noise reduction apparatus according to the ninth embodiment can operate in a manner similar to the noise reduction apparatus according to the sixth embodiment. That is, the structure utilizing the windowpane installed in the wall 4 can reduce the noise in the space surrounded by the wall 4.

As described above, according to the ninth embodiment, the loudspeaker is composed utilizing the sash 44 and the glass 45, and the driver 35 is built into the sash, whereby it is possible to place the noise reduction apparatus without causing the user to sense a discomfort at the sight of the loudspeaker on the wall. Also, the transparent film does not obstruct the light through the window or destroy the scenery viewed through the window.

Note that, also in the ninth embodiment, it is possible to perform the feed forward control as described in the seventh embodiment. Also, as described in the eighth embodiment, the structure by which the sound radiated into the space is detected by detecting the vibrations of the film may be used in place of using the error detector 2.

The noise reduction apparatus according to the present invention can be used as a sound insulator, or an apparatus for reducing noise passing through a wall. Also, the noise reduction apparatus according to the present invention reduces a sound in a position of a control point, whereby it is possible to reduce an audio signal as well as the noise. Thus, it is possible to use the noise reduction apparatus according to the present invention as an audio characteristic adjusting apparatus.

While the invention has been described in detail, the foregoing description is in all aspects illustrative and not restrictive. It is understood that numerous other modifications and variations can be devised without departing from the scope of the invention.

What is claimed is:

1. A noise reduction apparatus for reducing noise propagated toward a predetermined space on one side of a wall from an external noise source on another side of the wall, comprising:
a control sound source, which is placed on the wall so as to block a noise propagation path, for radiating a sound into the predetermined space;
a sound detector for detecting a sound propagated from the noise source through the control sound source; and
a control section for causing the control sound source to radiate a sound so as to minimize a sound to be detected by the sound detector, based on results detected by the sound detector.

2. The noise reduction apparatus according to claim 1, further comprising a housing, which is attached to the surface of the wall so as to face the noise source, for generating space for noise reduction between the housing and the wall; wherein
the control sound source is placed on the housing attached to the surface of the wall;
the sound detector is placed in the space for noise reduction; and
the control sound source radiates a sound into the space for noise reduction.

3. The noise reduction apparatus according to claim 2, wherein
a plurality of housings are attached to the surface of the wall adjacently to each other, and
the noise reduction apparatus further comprises a vibration damping section for damping a vibration in a position of a barycenter of each portion of the surface of the wall, which is divided by the plurality of housings having space for noise reduction.

4. The noise reduction apparatus according to claim 3, wherein the vibration damping section is a pole connecting the housing with the wall.

5. The noise reduction apparatus according to claim 4, wherein the sound detector is connected to the pole.

6. The noise reduction apparatus according to claim 3, wherein the vibration damping section is a plummet placed in the position of the barycenter.

7. The noise reduction apparatus according to claim 2, further comprising a film, which is connected to the housing, for generating a closed space between the film and the control sound source.

8. The noise reduction apparatus according to claim 2, wherein the control section is placed in the space for noise reduction.

9. The noise reduction apparatus according to claim 1, further comprising a noise detector placed outside the predetermined space for detecting the noise, wherein
the control section generates the control signal based on results detected by the sound detector and the noise detector.

10. The noise reduction apparatus according to claim 1, wherein the control sound source is a piezoelectric loudspeaker.

11. The noise reduction apparatus according to claim 1, wherein
the wall has a hole,
the control sound source includes:
a board connected to the wall so as to block the hole;
a vibrating component placed so as to face the predetermined space for forming an air layer with the board, and which is vibrated by a sound radiated into the air layer; and
a driver for radiating the sound into the air layer, and
the control section causes the driver to radiate the sound by the control signal.

12. The noise reduction apparatus according to claim 11, wherein the sound detector, which is placed in the predetermined space, detects the sound by detecting a sound pressure and a phase of the sound propagated toward the predetermined space.

13. The noise reduction apparatus according to claim 11, wherein the sound detector detects the sound propagated toward the predetermined space by detecting a vibration of the vibrating component.

14. The noise reduction apparatus according to claim 11, wherein the board and the vibrating component are made of a transparent material.
